SEQUENCE LISTING (12 sequences)

SEQ ID NO: 1 - protein, 10 aa, Artificial Sequence
  synthetic construct; MHC-I binding peptide
AAAAAAAALY

SEQ ID NO: 2 - protein, 9 aa, Artificial Sequence
  synthetic construct; MHC-I binding peptide
AAAAALQAK

SEQ ID NO: 3 - protein, 8 aa, Artificial Sequence
  synthetic construct; MHC-I binding peptide
AAAAALWL

SEQ ID NO: 4 - protein, 9 aa, Artificial Sequence
  synthetic construct; MHC-I binding peptide
AAAAARAAL

SEQ ID NO: 5 - protein, 9 aa, Artificial Sequence
  synthetic construct; MHC-I binding peptide
AAAAEEEEE

SEQ ID NO: 6 - protein, 9 aa, Artificial Sequence
  synthetic construct; MHC-I binding peptide
AAAAFEAAL

SEQ ID NO: 7 - protein, 9 aa, Artificial Sequence
  synthetic construct; MHC-I binding peptide
AAAAPYAGW

SEQ ID NO: 8 - protein, 9 aa, Artificial Sequence
  synthetic construct; MHC-I binding peptide
AAAARAAAL

SEQ ID NO: 9 - protein, 9 aa, Artificial Sequence
  synthetic construct; MHC-I binding peptide
AAAATCALV

SEQ ID NO: 10 - protein, 9 aa, Artificial Sequence
  synthetic construct; MHC-I binding peptide
AAADAAAAL

SEQ ID NO: 11 - protein, 9 aa, Artificial Sequence
  synthetic construct; MHC-I binding peptide
AAADFAHAE

SEQ ID NO: 12 - protein, 9 aa, Artificial Sequence
  synthetic construct; MHC-I binding peptide
AAADPKVAF

(19) United States
(12) Patent Application Publication
(10) Pub. No.: US 2019/0259474 A1
(43) Pub. Date: Aug. 22, 2019

(54) GAN-CNN FOR MHC PEPTIDE BINDING PREDICTION

(71) Applicant: REGENERON PHARMACEUTICALS, INC., Tarrytown, NY (US)

(72) Inventors: Xingjian Wang, Scarsdale, NY (US); Ying Huang, Ardsley, NY (US); Wei Wang, Elmsford, NY (US); Qi Zhao, Chappaqua, NY (US)

(21) Appl. No.: 16/278,611

(22) Filed: Feb. 18, 2019

Related U.S. Application Data
(60) Provisional application No. 62/631,710, filed on Feb. 17, 2018.

(51) Int. Cl. G16C 20/70; G16C 99/00; G16C 60/00; G16C 20/30; G16C 20/40; G16C 20/50; G16C 20/90

(52) U.S. Cl. CPC G16C 20/70; G16C 99/00; G16C 60/00; G16C 20/90; G16C 20/40; G16C 20/50; G16C 20/30

(57) ABSTRACT

Methods for training a generative adversarial network (GAN) in conjunction with a convolutional neural network (CNN) are disclosed. The GAN and the CNN can be trained using biological data, such as protein interaction data. The CNN can be used for identifying new data as positive or negative. Methods are disclosed for synthesizing a polypeptide associated with new protein interaction data identified as positive.

### CROSS-REFERENCE TO RELATED APPLICATIONS

This application claims benefit of U.S. Provisional Application No. 62/631,710, filed Feb. 17, 2018, which is hereby incorporated herein by reference in its entirety.

### REFERENCE TO SEQUENCE LISTING

The Sequence Listing submitted Feb. 18, 2019 as a text file named “37595_0028U2_Sequence Listing.txt,” created on Feb. 13, 2019, and having a size of 2,827 bytes is hereby incorporated by reference pursuant to 37 C.F.R. § 1.52(e)(5).

### BACKGROUND

One of the biggest issues facing the use of machine learning in is the lack of availability of large, annotated datasets. The annotation of data is not only expensive and time consuming but also highly dependent on the availability of expert observers. The limited amount of training data can inhibit the performance of supervised machine learning algorithms which often need very large quantities of data on which to train to avoid overfitting. So far, much effort has been directed at extracting as much information as possible from what data is available. One area in particular that suffers from lack of large, annotated datasets is analysis of biological data, such as protein interaction data. The ability to predict how proteins may interact is invaluable to the identification of new therapeutics.

Advances in immunotherapy are developing rapidly and are providing new medicines that modulate a patient's immune system to help fight diseases including cancer, autoimmune disorders, and infections. For example, checkpoint inhibitor molecules such as PD-1 and ligands of PD-1 have been identified that are used to develop drugs that inhibit or stimulate signal transduction through PD-1 and thereby modulate a patient's immune system. These new drugs have been very effective in some cases but not all. One reason in some 80 percent of cancer patients is that their tumors do not have enough cancer antigens to attract T cells.

Targeting an individual's tumor-specific mutations is attractive because these specific mutations generate tumor specific peptides, referred to as neoantigens, that are new to the immune system and are not found in normal tissues. Compared with tumor-associated self-antigens, neoantigens elicit T-cell responses not subject to host central tolerance in the thymus and also produce fewer toxicities arising from autoimmune reactions to non-malignant cells (Nature Biotechnology 35, 97 (2017).

The key question for neoepitope discovery is which mutated proteins are processed into 8- to 11-residue peptides by the proteasome, shuttled into the endoplasmic reticulum by the transporter associated with antigen processing (TAP) and loaded onto newly synthesized major histocompatibility complex class I (MHC-I) for recognition by CD8+ T cells (Nature Biotechnology 35, 97 (2017)).

Computational methods for predicting peptide interaction with MHC-I are known in the art. Although some computational methods focus on predicting what happens during antigen processing (e.g., NetChop) and peptide transport (e.g., NetCTL), most efforts focus on modeling which peptides bind to the MHC-I molecule. Neural network-based methods, such as NetMHC, are used to predict antigen sequences that generate epitopes fitting the groove of a patient's MHC-I molecules. Other filters can be applied to deprioritize hypothetical proteins and gauge whether a mutated amino acid either is likely orientated facing out of the MHC (toward the T-cell receptor) or reduces the affinity of the epitope for the MHC-I molecule itself (Nature Biotechnology 35, 97 (2017).

There are many reasons these predictions may be incorrect. Sequencing already introduces amplification biases and technical errors in the reads used as starting material for peptides. Modeling epitope processing and presentation also must take into account the fact that humans have ˜5,000 alleles encoding MHC-I molecules, with an individual patient expressing as many as six of them, all with different epitope affinities. Methods such as NetMHC typically require 50-100 experimentally determined peptide-binding measurements for a particular allele to build a model with sufficient accuracy. But as many MHC alleles lack such data, ‘pan-specific’ methods—capable of predicting binders based on whether MHC alleles with similar contact environments have similar binding specificities—have increasingly come to the fore.

Thus, there is a need for improved systems and methods for generating data sets for use in machine learning applications, particularly biological data sets. Peptide binding prediction techniques may benefit from such improved systems and methods. Therefore, it is an object of the invention to provide computer-implemented systems and methods that have improved capability generate data sets for training machine learning applications to make predictions, including predicting peptide binding to MHC-I.

### SUMMARY

It is to be understood that both the following general description and the following detailed description are exemplary and explanatory only and are not restrictive.

Methods and systems are disclosed for training a generative adversarial network (GAN), comprising, generating, by a GAN generator, increasingly accurate positive simulated data until a GAN discriminator classifies the positive simulated data as positive, presenting the positive simulated data, positive real data, and negative real data to a convolutional neural network (CNN), until the CNN classifies each type of data as positive or negative, presenting the positive real data and the negative real data to the CNN to generate prediction scores, determining, based on the prediction scores, whether the GAN is trained or not trained, and outputting the GAN and the CNN. The method may be repeated until the GAN is satisfactorily trained. The positive simulated data, the positive real data, and the negative real data comprise biological data. The biological data may comprise protein-protein interaction data. The biological data may comprise polypeptide-MHC-I interaction data. The positive simulated data may comprise positive simulated polypeptide-MHC-I interaction data, the positive real data comprises positive real polypeptide-MHC-I interaction data, and the negative real data comprises negative real polypeptide-MHC-I interaction data.

Additional advantages will be set forth in part in the description which follows or may be learned by practice. The advantages will be realized and attained by means of the elements and combinations particularly pointed out in the appended claims.

### BRIEF DESCRIPTION OF THE DRAWINGS

The accompanying drawings, which are incorporated in and constitute a part of this specification, illustrate embodiments and together with the description, serve to explain the principles of the methods and systems:

FIG. 1 is a flowchart of an example method.

FIG. 2 is an exemplary flow diagram showing a portion of a process of predicting peptide binding, including generating and training GAN models.

FIG. 3 is an exemplary flow diagram showing a portion of a process of predicting peptide binding, including generating data using trained GAN models and training CNN models.

FIG. 4 is an exemplary flow diagram showing a portion of a process of predicting peptide binding, including completing training CNN models and generating predictions of peptide binding using the trained CNN models.

FIG. 5A is an exemplary data flow diagram of a typical GAN.

FIG. 5B is an exemplary data flow diagram of a GAN generator.

FIG. 6 is an exemplary block diagram of a portion of processing stages included in a generator used in a GAN.

FIG. 7 is an exemplary block diagram of a portion of processing stages included in a generator used in a GAN.

FIG. 8 is an exemplary block diagram of a portion of processing stages included in a discriminator used in a GAN.

FIG. 9 is an exemplary block diagram of a portion of processing stages included in a discriminator used in a GAN.

FIG. 10 is a flowchart of an example method.

FIG. 11 is an exemplary block diagram of a computer system in which the processes and structures involved in predicting peptide binding may be implemented.

FIG. 12 is a table showing the results of the specified prediction models for predicting protein binding to MHC-I protein complex for the indicated HLA alleles.

FIG. 13A is table showing data used to compare prediction models.

FIG. 13B is a bar graph comparing AUC of our implementation of the same CNN architecture to that in Vang's paper.

FIG. 13C is a bar graph comparing the described implementation to existing systems.

FIG. 14 is a table showing bias obtained by choosing a biased test set.

FIG. 15 is a line graph of SRCC versus test size showing the smaller the test size, the better SRRC.

FIG. 16A is a table showing data used to compare Adam and RMSprop neural networks.

FIG. 16B is a bar graph comparing AUC between neural networks trained by Adam and RMSprop optimizer.

FIG. 16C is a bar graph comparing SRCC between neural networks trained by Adam and RMSprop optimizer.

FIG. 17 is a table showing that a mix of fake data and real data gets better prediction than fake data alone.

### DETAILED DESCRIPTION OF THE INVENTION

Before the present methods and systems are disclosed and described, it is to be understood that the methods and systems are not limited to specific methods, specific components, or to particular implementations. It is also to be understood that the terminology used herein is for the purpose of describing particular embodiments only and is not intended to be limiting.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other components, integers or steps. “Exemplary” means “an example of” and is not intended to convey an indication of a preferred or ideal embodiment. “Such as” is not used in a restrictive sense, but for explanatory purposes.

It is understood that the methods and systems are not limited to the particular methodology, protocols, and reagents described as these may vary. It is also to be understood that the terminology used herein is for the purpose of describing particular embodiments only, and is not intended to limit the scope of the present methods and system which will be limited only by the appended claims.

Unless defined otherwise, all technical and scientific terms used herein have the same meanings as commonly understood by one of skill in the art to which the methods and systems belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present methods and systems are not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Disclosed are components that can be used to perform the methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all embodiments of this application including, but not limited to, steps in methods. Thus, if there are a variety of additional steps that can be performed it is understood that each of these additional steps can be performed with any specific embodiment or combination of embodiments of the methods.

The present methods and systems may be understood more readily by reference to the following detailed description of preferred embodiments and the examples included therein and to the Figures and their previous and following description.

The methods and systems may take the form of an entirely hardware embodiment, an entirely software embodiment, or an embodiment combining software and hardware embodiments. Furthermore, the methods and systems may take the form of a computer program product on a computer-readable storage medium having computer-readable program instructions (e.g., computer software) embodied in the storage medium. More particularly, the present methods and systems may take the form of web-implemented computer software. Any suitable computer-readable storage medium may be utilized including hard disks, CD-ROMs, optical storage devices, or magnetic storage devices.

Embodiments of the methods and systems are described below with reference to block diagrams and flowchart illustrations of methods, systems, apparatuses and computer program products. It will be understood that each block of the block diagrams and flowchart illustrations, and combinations of blocks in the block diagrams and flowchart illustrations, respectively, can be implemented by computer program instructions. These computer program instructions may be loaded onto a general purpose computer, special purpose computer, or other programmable data processing apparatus to produce a machine, such that the instructions which execute on the computer or other programmable data processing apparatus create a means for implementing the functions specified in the flowchart block or blocks.

These computer program instructions may also be stored in a computer-readable memory that can direct a computer or other programmable data processing apparatus to function in a particular manner, such that the instructions stored in the computer-readable memory produce an article of manufacture including computer-readable instructions for implementing the function specified in the flowchart block or blocks. The computer program instructions may also be loaded onto a computer or other programmable data processing apparatus to cause a series of operational steps to be performed on the computer or other programmable apparatus to produce a computer-implemented process such that the instructions that execute on the computer or other programmable apparatus provide steps for implementing the functions specified in the flowchart block or blocks.

Accordingly, blocks of the block diagrams and flowchart illustrations support combinations of means for performing the specified functions, combinations of steps for performing the specified functions and program instruction means for performing the specified functions. It will also be understood that each block of the block diagrams and flowchart illustrations, and combinations of blocks in the block diagrams and flowchart illustrations, can be implemented by special purpose hardware-based computer systems that perform the specified functions or steps, or combinations of special purpose hardware and computer instructions.

### I. Definitions

The abbreviation “SRCC” refers to Spearman's Rank Correlation Coefficient (SRCC) Calculations.

The term “ROC curve” refers to a receiver operating characteristic curve.

The abbreviation “CNN” refers to a convolutional neural network.

The abbreviation “GAN” refers to a generative adversarial network.

The term “HLA” refers to human leukocyte antigen. The HLA system or complex is a gene complex encoding the major histocompatibility complex (MEW) proteins in humans. The major HLA class I genes are HLA-A, HLA-B, and HLA-C, while HLA-E, HLA-F, and HLA-G are the minor genes.

The term “MHC I” or “major histocompatibility complex I” refers to a set of cell surface proteins composed of an α chain having three domains—α1, α2, and α3. The α3 domain is a transmembrane domain while the α1 and α2 domains are responsible for forming a peptide-binding groove.

The “polypeptide-MHC I interaction” refers to the binding of a polypeptide in the peptide-binding groove of the MEW I.

As used herein, “biological data” means any data derived from measuring biological conditions of human, animals or other biological organisms including microorganisms, viruses, plants and other living organisms. The measurements may be made by any tests, assays or observations that are known to physicians, scientists, diagnosticians, or the like. Biological data may include, but is not limited to, DNA sequences, RNA sequence, protein sequences, protein interactions, clinical tests and observations, physical and chemical measurements, genomic determinations, proteomic determinations, drug levels, hormonal and immunological tests, neurochemical or neuro-physical measurements, mineral and vitamin level determinations, genetic and familial histories, and other determinations that may give insight into the state of the individual or individuals that are undergoing testing. Herein, the use of the term “data” is used interchangeably with “biological data”.

### II. Systems for Predicting Peptide Binding

One embodiment of the present invention provides a system for predicting peptide binding to MHC-I that has a generative adversarial network (GAN)-convolutional neural network (CNN) framework, also referred to as a Deep Convolutional Generative Adversarial Network. The GAN contains a CNN discriminator and a CNN generator, and can be trained on existing peptide-MHC-I binding data. The disclosed GAN-CNN systems have several advantages over existing systems for predicting peptide-MHC-I binding including, but not limited to, the ability to be trained on unlimited alleles and better prediction performance. The present methods and systems, while described herein with regard to predicting peptide binding to MHC-I, the applications of the methods and systems are not so limited. Predicting peptide binding to MHC-I is provided as an example application of the improved GAN-CNN system described herein. The improved GAN-CNN system is applicable to a wide variety of biological data to generate various predictions.

### A. Exemplary Neural Network Systems and Methods

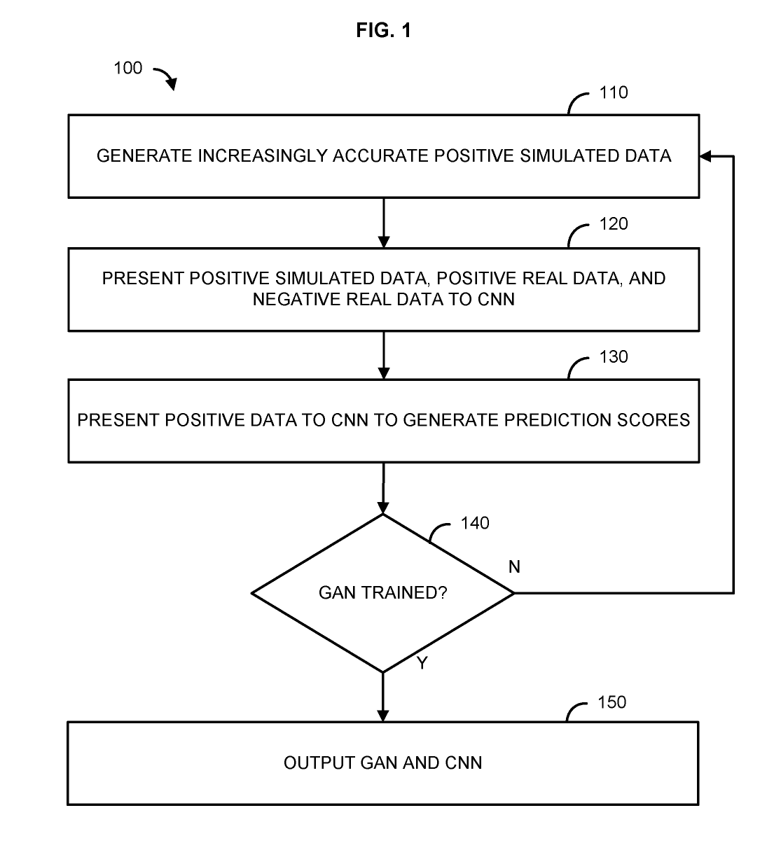

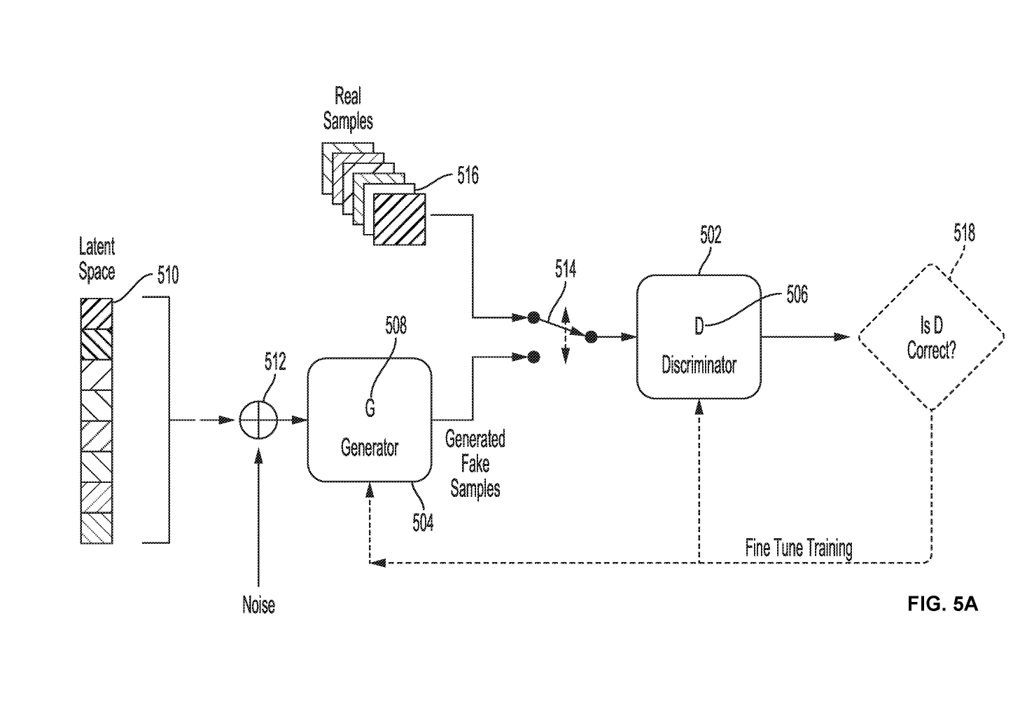

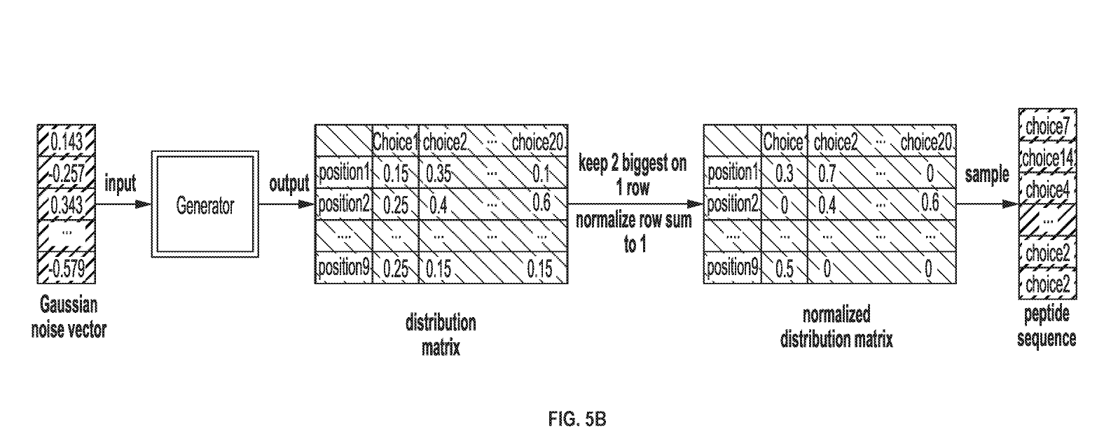

FIG. 1 is a flowchart 100 of an example method. Beginning with step 110, increasingly accurate positive simulated data can be generated by a generator (see 504 of FIG. 5A) of a GAN. The positive simulated data may comprise biological data, such as protein interaction data (e.g., binding affinity). Binding affinity is one example of a measure of the strength of the binding interaction between a biomolecule (e.g., protein, DNA, drug, etc. . . . ) to biomolecule (e.g., protein, DNA, drug, etc. . . . ). Binding affinity may be expressed numerically as a half maximal inhibitory concentration (IC₅₀) value. A lower number indicates a higher affinity. Peptides with IC50 values<50 nM are considered high affinity, <500 nM is intermediate affinity and <5000 nM is low affinity. IC₅₀ may be transformed into a binding category as binding (1) or not binding (−1).

The positive simulated data may comprise positive simulated polypeptide-MHC-I interaction data. Generating positive simulated polypeptide-MHC-I interaction data can be based, at least in part, on real polypeptide-MHC-I interaction data. Protein interaction data may comprise a binding affinity score (e.g., IC₅₀, binding category) representing a likelihood that two proteins will bind. Protein interaction data, such as polypeptide-MHC-I interaction data, may be received from, for example, any number of databases such as PepBDB, PepBind, the Protein Data Bank, the Biomolecular Interaction Network Database (BIND), Cellzome (Heidelberg, Germany), the Database of Interacting Proteins (DIP), Dana Farber Cancer Institute (Boston, Mass., USA), the Human Protein Reference Database (HPRD), Hybrigenics (Paris, France), the European Bioinformatics Institute's (EMBL-EBI, Hinxton, UK) IntAct, the Molecular Interactions (MINT, Rome, Italy) database, the Protein-Protein Interaction Database (PPID, Edinburgh, UK) and the Search Tool for the Retrieval of Interacting Genes/Proteins (STRING, EMBL, Heidelberg, Germany), and the like. Protein interaction data may be stored in a data structure comprising one or more of, a particular polypeptide sequence as well as an indication regarding the interaction of the polypeptides (e.g., the interaction between the polypeptide sequence and MHC-I). In an embodiment, the data structure may conform to the HUPO PSI Molecular Interaction (PSI MI) Format, which may comprise one or more entries, wherein an entry describes one or more protein interactions. The data structure may indicate the source of the entry, for example, a data provider. A release number and a release date assigned by the data provider may be indicated. An availability list may provide statements on the availability of the data. An experiment list may indicate experiment descriptions including at least one set of experimental parameters, usually associated with a single publication. In large-scale experiments, normally only one parameter, often the bait (protein of interest), is varied across a series of experiments. The PSI MI format may indicate both constant parameters (e.g., experimental technique) and variable parameters (e.g., the bait). An interactor list may indicate a set of interactors (e.g., proteins, small molecules, etc. . . . ) participating in an interaction. A protein interactor element may indicate a “normal” form of a protein commonly found in databases like Swiss-Prot and TrEMBL, which may include data, such as name, cross-references, organism, and amino acid sequence. An interaction list may indicate one or more interaction elements. Each interaction may indicate an availability description (a description of the data availability), and a description of the experimental conditions under which it has been determined. An interaction may also indicate a confidence attribute. Different measures of confidence in an interaction have been developed, for example, the paralogous everification method, and the Protein Interaction Map (PIM) biological score. Each interaction may indicate a participant list containing two or more protein participant elements (that is, the proteins participating in the interaction). Each protein participant element may include a description of the molecule in its native form and/or the specific form of the molecule in which it participated in the interaction. A feature list may indicate sequence features of the protein, for example, binding domains or post-translational modifications relevant for the interaction. A role may be indicated that describes the particular role of the protein in the experiment—for example, whether the protein was a bait or prey. Some or all of the preceding elements may be stored in the data structure. An example data structure is may be an XML file, for example:

<entry>
<interactorList>
<Interactor id=“Succinate>
<names>
<shortLabel>Succinate</shortLabel>
<fullName>Succinate</fullName>
</names>
</Interactor>
</interactorList>
<interactionList>
<interaction>
<names>
<shortLabel> Succinate dehydrogenas
catalysis </shortLabel>
<fullName>Interaction between </fullName>
</names>
<participantList>
<Participant>
<proteinInteractorRef ref=“Succinate”/>
<biologicalrole>neutral</role>
</proteinParticipant> <proteinParticipant>
<proteinInteractorRef ref=“Fumarate”/>
<role>neutral</role> </proteinParticipant>
<proteinParticipant> <proteinInteractorRef
ref=“Succdeh”/> <role>neutral</role>
</proteinParticipant> </participantList>
</interaction>
</interactionList>

The GAN can include, for example, a Deep Convolutional GAN (DCGAN).

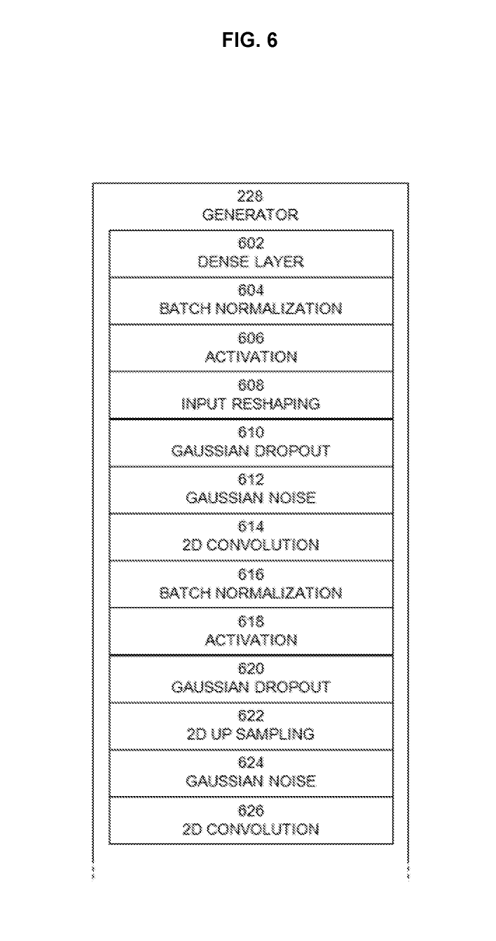

Referring to FIG. 5A, an example of a basic structure of a GAN is shown. A GAN is essentially a way of training a neural network. GANs typically contain two independent neural networks, discriminator 502 and generator 504, that work independently and may act as adversaries. Discriminator 502 may be a neural network that is to be trained using training data generated by generator 504. Discriminator 502 may include a classifier 506 that may be trained to perform the task of discriminating among data samples. Generator 504 may generate random data samples that resemble real samples, but which may be generated including, or may be modified to include, features that render them as fake or artificial samples. The neural networks included discriminator 502 and generator 504 may typically be implemented by multi-layer networks consisting of a plurality of processing layers, such as dense processing, batch normalization processing, activation processing, input reshaping processing, gaussian dropout processing, gaussian noise processing, two-dimensional convolution, and two-dimensional up sampling. This is shown in more detail in FIG. 6-FIG. 9 below.

For example, classifier 506 may be designed to identify data samples indicating various features. Generator 504 may include an adversary function 508 that may generate data intended to fool discriminator 502 using data samples that are almost, but not quite, correct. For example, this may be done by picking a legitimate sample randomly from a training set 510 (latent space) and synthesizing a data sample (data space) by randomly altering its features, such as by adding random noise 512. The generator network, G, may be considered to be a mapping from some the latent space, to the data space. This may be expressed formally as G:G(z)→R^{|x|}, where z∈R^{|x|} is a sample from the latent space, x∈R^{|x|} is a sample from the data space, and denotes the number of dimensions.

The discriminator network, D, may be considered to be a mapping from data space to a probability that the data (e.g., peptide) is from the real data set, rather than the generated (fake or artificial) data set. This may be expressed formally as: D:D(x)→(0; 1). During training, discriminator 502 may be presented, by randomizer 514, with a random mix of legitimate data samples 516 from real training data, along with fake or artificial (e.g., simulated) data samples generated by generator 504. For each data sample, discriminator 502 may attempt to identify legitimate and fake or artificial inputs, yielding result 518. For example, for a fixed generator, G, the discriminator, D, may be trained to classify data (e.g., peptides) as either being from the training data (real, close to 1) or from a fixed generator (simulated, close to 0). For each data sample, discriminator 502 may further attempt to identify positive or negative inputs (regardless of whether the input is simulated or real), yielding result 518.

Based on the series of results 518, both discriminator 502 and generator 504 may attempt to fine-tune their parameters to improve their operation. For example, if discriminator 502 makes the right prediction, generator 504 may update its parameters in order to generate better simulated samples to fool discriminator 502. If discriminator 502 makes an incorrect prediction, discriminator 502 may learn from its mistake to avoid similar mistakes. Thus, the updating of discriminator 502 and generator 504 may involve a feedback process. This feedback process may be continuous or incremental. The generator 504 and the discriminator 502 may be iteratively executed in order to optimize data generation and data classification. In an incremental feedback process, the state of generator 504 is frozen and discriminator 502 is trained until an equilibrium is established and training of discriminator 502 is optimized. For example, for a given frozen state of generator 504, discriminator 502 may be trained so that is it optimized with respect to the state of generator 504. Then, this optimized state of discriminator 502 may be frozen and generator 504 may be trained so as to lower the accuracy of the discriminator to some predetermined threshold. Then, the state of generator 504 may be frozen and discriminator 502 may be trained, and so on.

In a continuous feedback process, the discriminator may not be trained until its state is optimized, but rather may only be trained for one or a small number of iterations, and the generator may be updated simultaneously with the discriminator.

If the generated simulated data set distribution is able to match the real data set distribution perfectly, then the discriminator will be maximally confused and cannot distinguish real samples from fake ones (predicting 0.5 for all inputs).

Returning to FIG. 1 at 110, generating the increasingly accurate positive simulated polypeptide-MHC-I interaction data can be performed (e.g., by the generator 504) until the discriminator 502 of the GAN classifies the positive simulated polypeptide-MHC-I interaction data as positive. In another aspect, generating the increasingly accurate positive simulated polypeptide-MHC-I interaction data can be performed (e.g., by the generator 504) until the discriminator 502 of the GAN classifies the positive simulated polypeptide-MHC-I interaction data as real positive. For example, the generator 504 can generate the increasingly accurate positive simulated polypeptide-MHC-I interaction data by generating a first simulated dataset comprising positive simulated polypeptide-MHC-I interactions for a MHC allele. The first simulated dataset can be generated according to one or more GAN parameters. The GAN parameters can comprise, for example, one or more of an allele type (e.g., HLA-A, HLA-B, HLA-C, or a subtype thereof), an allele length (e.g., from about 8 to 12 amino acids, from about 9 to 11 amino acids), a generating category, a model complexity, a learning rate, a batch size, or another parameter.

FIG. 5B is an exemplary data flow diagram of a GAN generator configured for generating positive simulated polypeptide-MHC-I interaction data for a MEW allele. As shown in FIG. 5B, a Gaussian noise vector can be input into the generator that outputs a distribution matrix. The input noises sampled from Gaussian provides variability that mimics different binding patterns. The output distribution matrix represents probability distribution of choosing each amino acid for every position in a peptide sequence. The distribution matrix can be normalized to get rid of choices that are less likely to provide binding signals and a specific peptide sequence can be sampled from the normalized distribution matrix.

The first simulated dataset can then be combined with positive real polypeptide interaction data, and/or negative real polypeptide interaction data (or a combination thereof) for the MHC allele to create a GAN training set. The discriminator 502 can then determine (e.g., according to a decision boundary) whether a polypeptide-MHC-I interaction for the MHC allele in the GAN training dataset is positive or negative and/or simulated or real. Based on the accuracy of the determination performed by the discriminator 502 (e.g., whether the discriminator 502 correctly identified the polypeptide-MHC-I interaction as positive or negative and/or simulated or real), one or more of the GAN parameters or the decision boundary can be adjusted. For example, one or more of the GAN parameters of the decision boundary can be adjusted to optimize the discriminator 502 in order to increase a likelihood of giving a high probability to positive real polypeptide-MHC-I interaction data, a low probability to the positive simulated polypeptide-MHC-I interaction data, and/or a low probability to the negative real polypeptide-MHC-I interaction data. One or more of the GAN parameters of the decision boundary can be adjusted to optimize the generator 504 in order to increase a probability of the positive simulated polypeptide-MHC-I interaction data being rated highly.

The process of generating the first simulated dataset, combining the first dataset with positive real polypeptide interaction data, and/or negative real polypeptide interaction data to generate a GAN training dataset, determining by the discriminator, and adjusting the GAN parameters and/or the decision boundary can be repeated until a first stop criterion is satisfied. For example, it can be determined whether the first stop criterion is satisfied by evaluating a gradient descent expression for the generator 504. As another example, it can be determined whether the first stop criterion is satisfied by evaluating a means squared error (MSE) function:

$$MSE = \frac{1}{n} {\sum }_{i = 1}^{n} {( {Y}_{i} - {\hat{Y}}_{i} )}^{2}$$

As another example, it can be determined whether the first stop criterion is satisfied by evaluating whether gradient is large enough to continue meaningful training. Because the generator 504 is updated by back propagation algorithm, each layer of a generator will have one or more gradients, for example, given a graph with 2 layers and each layer has 3 nodes, the output of the graph 1 is 1 dimensional (a scalar) and data is 2 dimensional. In this graph, the 1^{st} layer has 2*3=6 edges(w111, w112, w121, w122, w131, w132) connecting to data, and w111*data1+w112*data2=net11, and a sigmoid activation function may be used to get output o11=sigmoid(net11), similarly o12, o13 may be obtained, which form the output of 1^{st} layer; the 2^{nd} layer has 3*3=9 edges (w211, w212, w213, w221, w222, w223, w231, w232, w233) connecting to the 1^{st} layer outputs, and the 2^{nd} layer output is o21, o22, o23 and it connects to the final output with 3 edges, which is w311, w312, w313.

Each w in this graph has a gradient (an instruction of how to update w, essentially a number to be added), the number may be calculated by an algorithm referred to as Backpropagation following the idea of changing a parameter to the direction where loss (MSE) decreases, which is:

$$\frac{\partial E}{\partial {w}_{ij}} = \frac{\partial E}{\partial {o}_{j}} \frac{\partial {o}_{j}}{\partial {net}_{j}} \frac{\partial {net}_{j}}{\partial {w}_{ij}}$$

Where E is the MSE error, wᵢⱼ is the ith parameter on jth layer. Oⱼ is the output on jth layer, netⱼ is the before activation, the multiplication result on jth layer. And if the value de/dwᵢⱼ (gradient) for wᵢⱼ is not sufficiently large, the result is the training is not bringing changes for wᵢⱼ of the generator 504, and training should discontinue.

Next, after the GAN discriminator 502 classifies the positive simulated data (e.g., the positive simulated polypeptide-MHC-I interaction data) as positive and/or real, at step 120, the positive simulated data, positive real data, and/or negative real data (or a combination thereof) can be presented to a CNN until the CNN classifies each type of data as positive or negative. The positive simulated data, the positive real data, and/or the negative real data may comprise biological data. The positive simulated data may comprise positive simulated polypeptide-MHC-I interaction data. The positive real data may comprise positive real polypeptide-MHC-I interaction data. The negative real data may comprise negative real polypeptide-MHC-I interaction data. The data being classified may comprise polypeptide-MHC-I interaction data. Each of the positive simulated polypeptide-MHC-I interaction data, positive real polypeptide-MHC-I interaction data, and negative real polypeptide-MHC-I interaction data can be associated with a selected allele. For example, the selected allele can be selected from the group consisting of A0201, A202, A203, B2703, B2705, and combinations thereof.

Presenting the positive simulated polypeptide-MHC-I interaction data, positive real polypeptide-MHC-I interaction data, and negative real polypeptide-MHC-I interaction data to the CNN can include generating, e.g. by the generator 504 according to the set of GAN parameters, a second simulated data set comprising positive simulated polypeptide-MHC-I interactions for the WIC allele. The second simulated data set can be combined with positive real polypeptide interaction data, and/or negative real polypeptide interaction data (or a combination thereof) for the MHC allele to create a CNN training dataset.

The CNN training dataset can then be presented to the CNN to train the CNN. The CNN can then classify, according to one or more CNN parameters, a polypeptide-MHC-I interaction as positive or negative. This can include performing, by the CNN, a convolutional procedure, performing a Non Linearity (e.g., ReLu) procedure, performing a pooling or Sub Sampling procedure and/or performing a Classification (e.g., Fully Connected Layer) procedure.

Based on the accuracy of the classification by the CNN, one or more of the CNN parameters can be adjusted. The process of generating the second simulated data set, generating the CNN training dataset, classifying the polypeptide-MHC-I interaction, and adjusting the one or more CNN parameters can be repeated until a second stop criterion is satisfied. For example, it can be determined whether the second stop criterion is satisfied by evaluating a mean squared error (MSE) function.

Next, at step 130, the positive real data and/or negative real data can be presented to the CNN to generate prediction scores. The positive real data, and/or the negative real data may comprise biological data, such a protein interaction data including for example, binding affinity data. The positive real data may comprise positive real polypeptide-MHC-I interaction data. The negative real data may comprise negative real polypeptide-MHC-I interaction data. The prediction scores may be binding affinity scores. The prediction scores can comprise a probability of the positive real polypeptide-MHC-I interaction data being classified as positive polypeptide-MHC-I interaction data. This can include presenting the CNN with the real dataset and classifying, by the CNN according to the CNN parameters, a polypeptide-MHC-I interaction for the MHC allele as positive or negative.

At step 140 it can be determined whether the GAN is trained based on the prediction scores. This can include determining whether the GAN is trained by determining the accuracy of the CNN based on the prediction scores. For example, the GAN can be determined as trained if a third stop criterion is satisfied. Determining whether the third stop criterion is satisfied can comprise determining if an area under the curve (AUC) function is satisfied. Determining if the GAN is trained can comprise comparing one or more of the prediction scores to a threshold. If the GAN is trained as determined in step 140 then the GAN can optionally be output in step 150. If the GAN is not determined to be trained, the GAN can return to step 110.

Having trained the CNN and the GAN, a dataset (e.g., an unclassified dataset) can be presented to the CNN. The dataset can comprise unclassified biological data, such as unclassified protein interaction data. The biological data can comprise a plurality of candidate polypeptide-MHC-I interactions. The CNN can generate a predicted binding affinity and/or classify each of the candidate polypeptide-MHC-I interactions as positive or negative. A polypeptide can then be synthesized using those of the candidate polypeptide-MHC-I interactions classified as positive. For example, the polypeptide can comprise a tumor specific antigen. As another example, the polypeptide can comprise an amino acid sequence that specifically binds to an MHC-I protein encoded by a selected MHC allele.

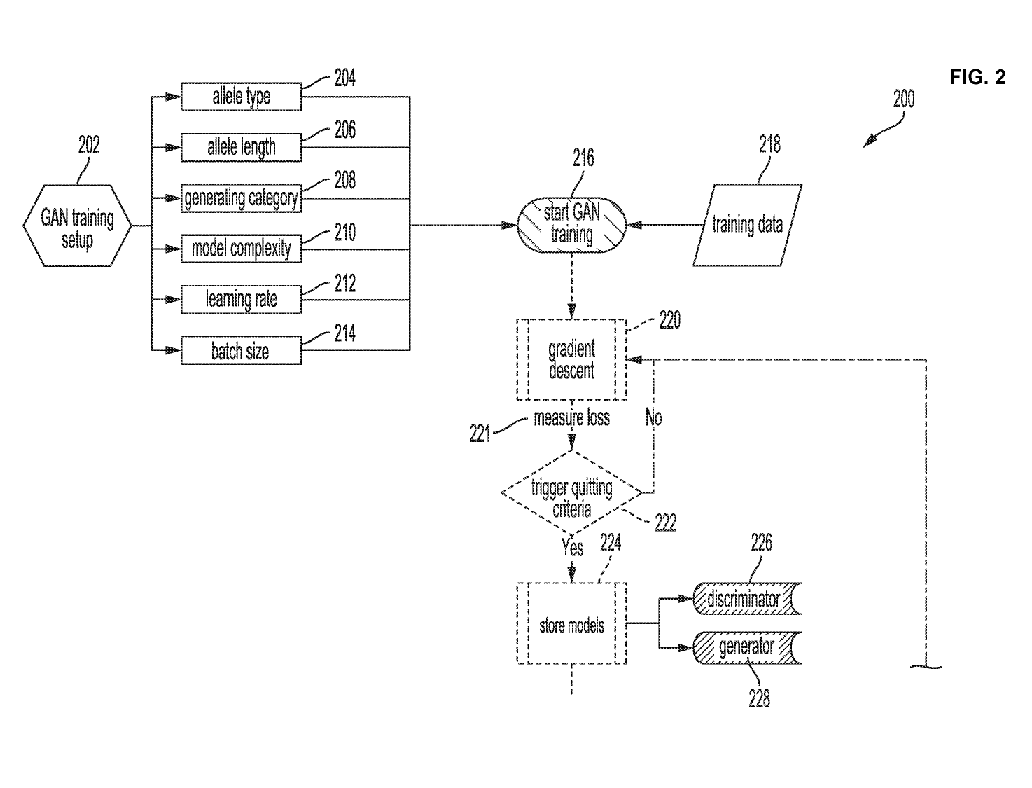

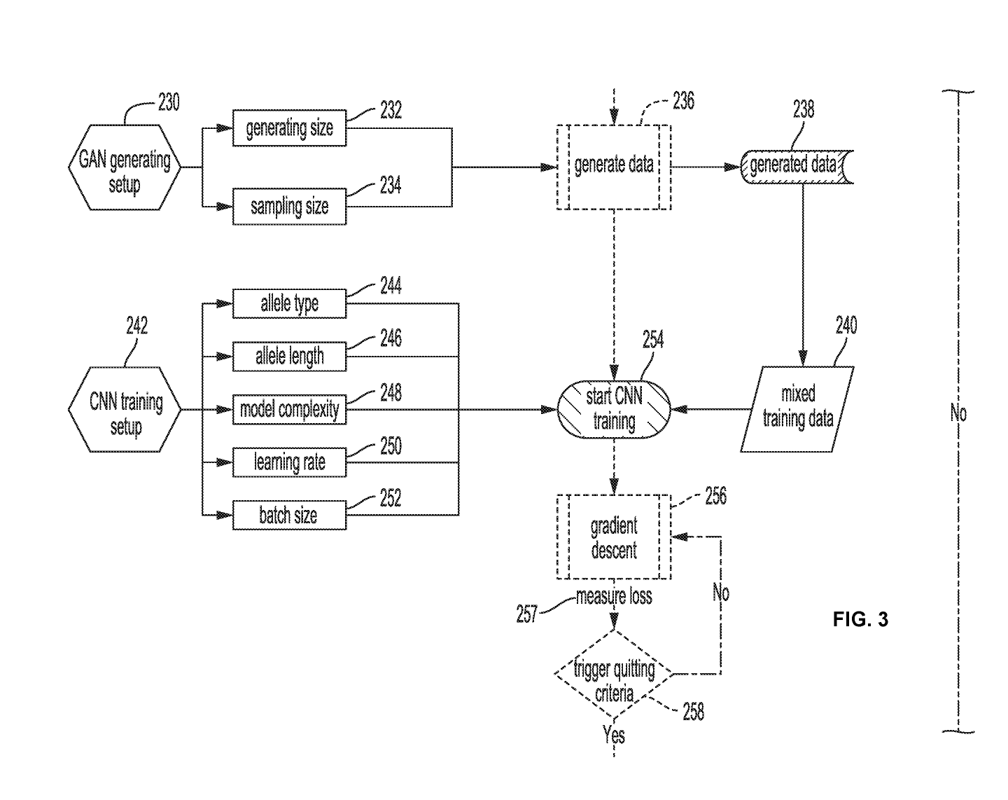

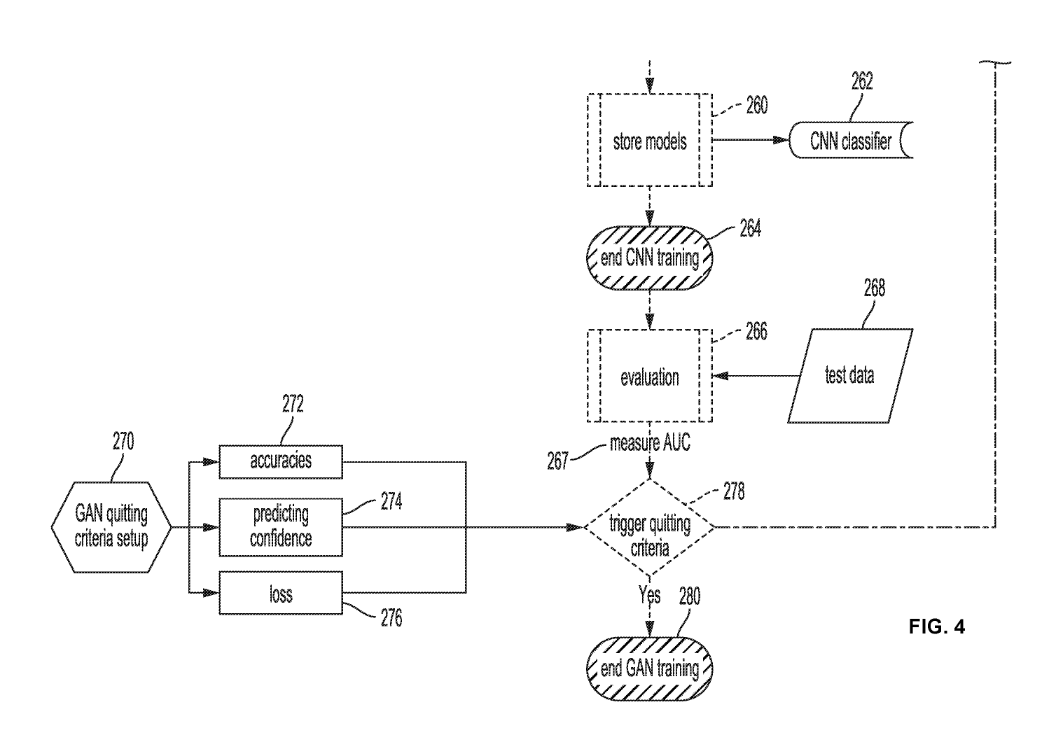

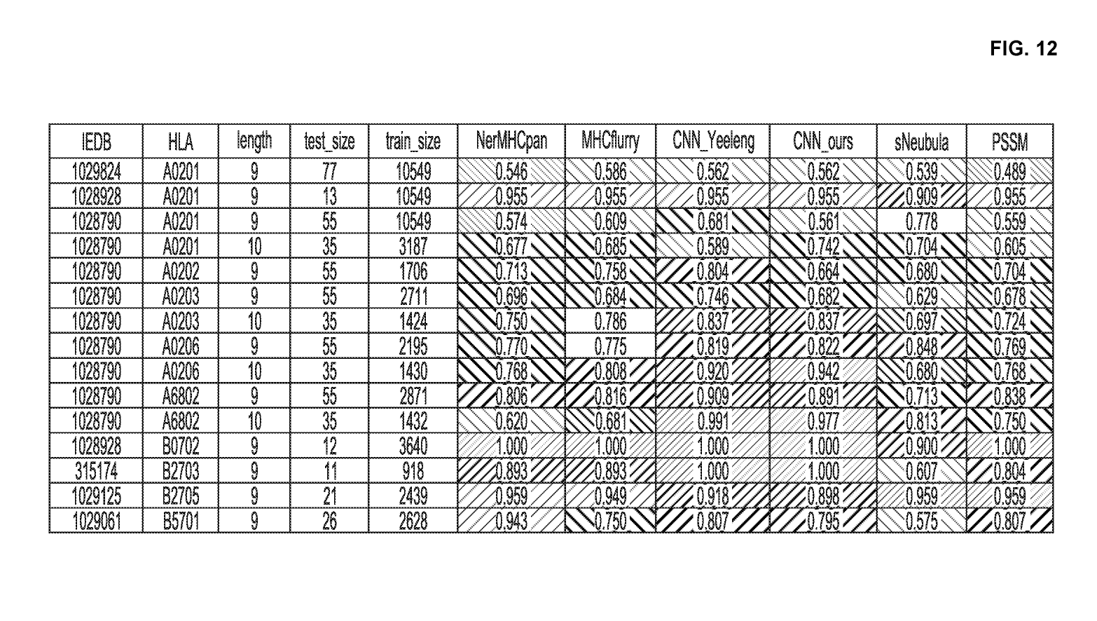

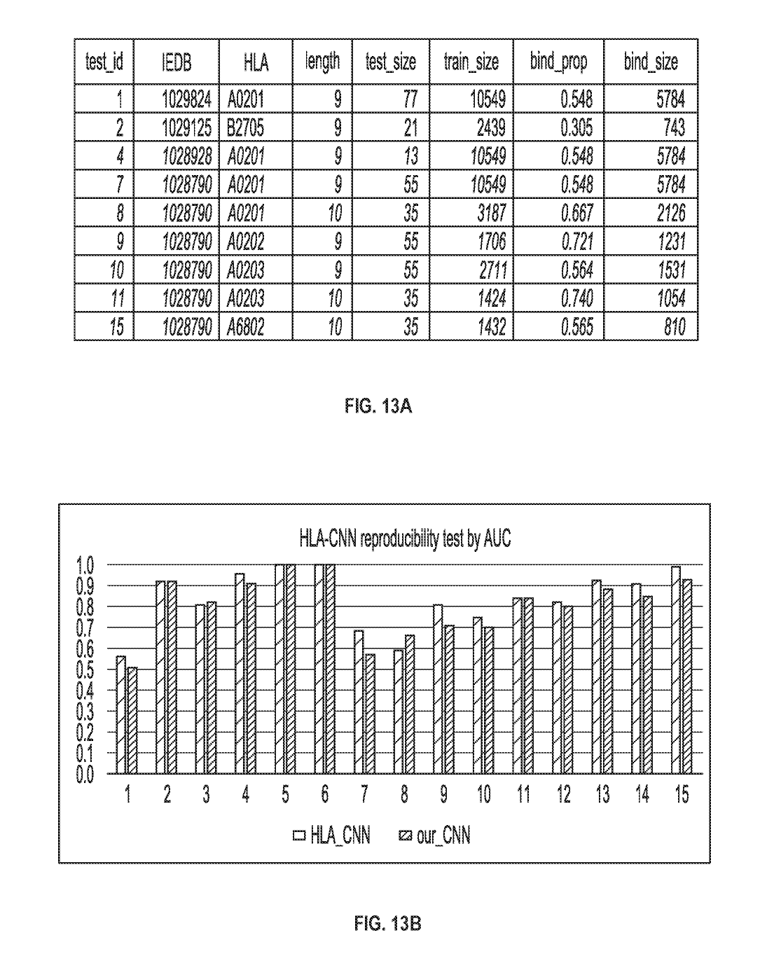

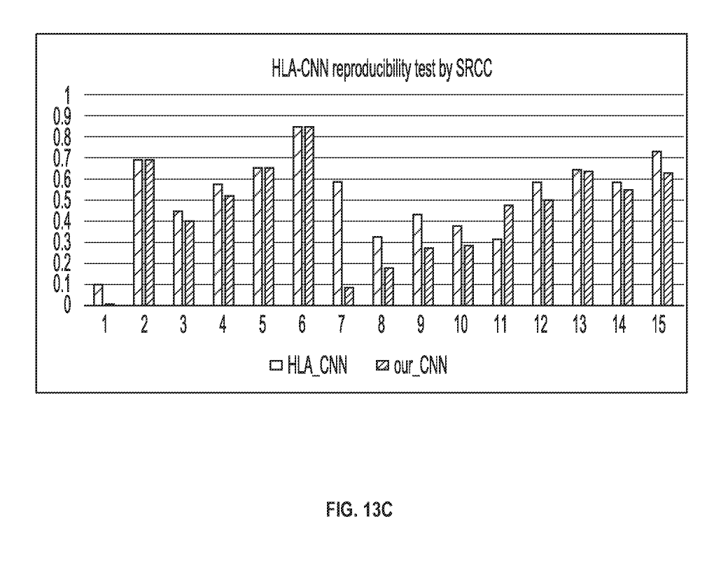

A more detailed exemplary flow diagram of a process 200 of prediction using a generative adversarial network (GAN) is shown in FIG. 2-FIG. 4. 202-214 generally correspond to 110, shown in FIG. 1. Process 200 may begin with 202, in which the GAN training is setup, for example, by setting a number of parameters 204-214 to control GAN training 216. Examples of parameters that may be set may include allele type 204, allele length 206, generating category 208, model complexity 210, learning rate 212, and batch size 214. Allele type parameters 204 may provide the capability to specify one or more allele types to be included in the GAN processing. Examples of such allele types are shown in FIG. 12. For example, specified alleles may include A0201, A0202, A0203, B2703, B2705, etc., shown in FIG. 12. Allele length parameters 206 may provide the capability to specify lengths of peptides that may bind to each specified allele type 204. Examples of such lengths are shown in FIG. 13. For example, for A0201 the specified length is shown as 9, or 10, for A0202 the specified length is shown as 9, for A0203 the specified length is shown as 9, or 10, for B2705 the specified length is shown as 9, etc. Generating category parameters 208 may provide the capability to specify categories of data to be generated during GAN training 216. For example, binding/non-binding categories may be specified. A collection of parameters corresponding to model complexity 210 may provide the capability to specify aspects of the complexity of the models to be used during GAN training 216. Examples of such aspects may include the number of layers, the number of nodes per layer, the window size for each convolutional layer, etc. Learning rate parameters 212 may provide the capability to specify one or more rates at which the learning processing performed in GAN training 216 is to converge. Examples of such learning rate parameters may include 0.0015, 0.015, 0.01, which are unitless values specifying relative rates of learning. Batch size parameters 214 may provide the capability to specify sizes of batches of training data 218 to be processed during GAN training 216. Examples of such batch sizes may include batches having 64 or 128 data samples. GAN training setup processing 202 may gather training parameters 204-214, process them to be compatible with GAN training 216 and input the processed parameters to GAN training 216 or store the processed parameters in the appropriate files or locations for use by GAN training 216.

At 216, GAN training may be started. 216-228 also generally correspond to 110, shown in FIG. 1. GAN training 216 may ingest training data 218, for example, in batches as specified by batch size parameters 214. Training data 218 may include data representing peptides with different binding affinity designations (bind or not) for MHC-I protein complexes encoded by different allele types, such as HLA allele types, etc. For example, such training data may include information relating to positive/negative MHC-peptide interaction binning and selection. Training data can comprise one or more of positive simulated polypeptide-MHC-I interaction data, positive real polypeptide-MHC-I interaction data, and/or negative real polypeptide-MHC-I interaction data.

At 220, a gradient descent process may be applied to the ingested training data 218. Gradient descent is an iterative process for performing machine learning, such as finding a minimum, or local minimum, of a function. For example, to find a minimum, or local minimum, of a function using gradient descent, variable values are updated in steps proportional to the negative of the gradient (or of the approximate gradient) of the function at the current point. For machine learning, a parameter space may be searched using gradient descent. Different Gradient Descent Strategies may find different “destinations” in parameter space so as to limit the predicted errors to an acceptable degree. In embodiments, a gradient descent process may adapt the learning rate to the input parameters, for example, performing larger updates for infrequent parameters and smaller updates for frequent parameters. Such embodiments may be suited for dealing with sparse data. For example, a gradient descent strategy known as RMSprop may provide improved performance with peptide binding datasets.

At 221 a loss measure may be applied to measure the loss or “cost” of processing. Examples of such loss measures may include Mean Squared Error, or cross entropy.

At 222, it may be determined whether or not quitting criteria for the gradient descent have been triggered. As gradient descent is an iterative process, criteria may be specified to determine when the iterative process should stop indicating that the generator 228 is capable of generating positive simulated polypeptide-MHC-I interaction data that is classified as positive and/or real by the discriminator 226. At 222, if it is determined that quitting criteria for the gradient descent have not been triggered, then the process may loop back to 220, and the gradient descent process continues. At 222, if it is determined that quitting criteria for the gradient descent have been triggered, then the process may continue with 224, in which the discriminator 226 and generator 228 may be trained, for example as described with reference to FIG. 5A. At 224, trained models for discriminator 226 and generator 228 may be stored. These stored models may include data defining the structure and coefficients that make up the models for discriminator 226 and generator 228. The stored models provide the capability to use generator 228 to generate artificial data and discriminator 226 to identify data, and when properly trained, provide accurate and useful results from discriminator 226 and generator 228.

The process may then continue with 230-238, which generally correspond to 120, shown in FIG. 1. At 230-238, generated data samples (e.g., positive simulated polypeptide-MHC-I interaction data) may be produced using the trained generator 228. For example, at 230, the GAN generating process may be setup, for example, by setting a number of parameters 232, 234 to control GAN generating 236. Examples of parameters that may be set may include generating size 232 and sampling size 234. Generating size parameters 232 may provide the capability to specify the size of the dataset to be generated. For example, the generated (positive simulated polypeptide-MHC-I interaction data) dataset size may be set to be 2.5 times the size of the real data (positive real polypeptide-MHC-I interaction data and/or negative real polypeptide-MHC-I interaction data). In this example, if the original real data in a batch is 64, then the corresponding generated simulated data in the batch is 160. Sampling size parameters 234 may provide the capability to specify the size of the sampling to be used in order to generate the dataset. For example, this parameter may be specified as the cutoff percentile of 20 amino acid choices in the final layer of the generator. As an example, specification of the 90th percentile means that all points less than 90th percentile will be set to 0, and the rest may be normalized using a normalizing function, such as a normalized exponential (softmax) function. At 236, trained generator 228 may be used to generate a dataset 236 that may be used to train a CNN model.

At 240, simulated data samples 238 produced by trained generator 228 and real data samples from the original dataset may be mixed to form a new set of training data 240, as generally corresponds to 120, shown in FIG. 1. Training data 240 can comprise one or more of positive simulated polypeptide-MHC-I interaction data, positive real polypeptide-MHC-I interaction data, and/or negative real polypeptide-MHC-I interaction data. At 242-262, a convolutional neural network (CNN) classifier model 262 may be trained using mixed training data 240. At 242, the CNN training may be setup, for example, by setting a number of parameters 244-252 to control CNN training 254. Examples of parameters that may be set may include allele type 244, allele length 246, model complexity 248, learning rate 250, and batch size 252. Allele type parameters 244 may provide the capability to specify one or more allele types to be included in the CNN processing. Examples of such allele types are shown in FIG. 12. For example, specified alleles may include A0201, A0202, B2703, B2705, etc., shown in FIG. 12. Allele length parameters 246 may provide the capability to specify lengths of peptides that may bind to each specified allele type 244. Examples of such lengths are shown in FIG. 13A. For example, for A0201 the specified length is shown as 9, or 10, for A0202 the specified length is shown as 9, for B2705 the specified length is shown as 9, etc. A collection of parameters corresponding to model complexity 248 may provide the capability to specify aspects of the complexity of the models to be used during CNN training 254. Examples of such aspects may include the number of layers, the number of nodes per layer, the window size for each convolutional layer, etc. Learning rate parameters 250 may provide the capability to specify one or more rates at which the learning processing performed in CNN training 254 is to converge. Examples of such learning rate parameters may include 0.001, which is a unitless parameter specifying a relative learning rate. Batch size parameters 252 may provide the capability to specify sizes of batches of training data 240 to be processed during CNN training 254. For example, if the training dataset is divided into 100 equal pieces, the batch size may be an integer form of the training data size (train_data_size)/100. CNN training setup processing 242 may gather training parameters 244-252, process them to be compatible with CNN training 254 and input the processed parameters to CNN training 254 or store the processed parameters in the appropriate files or locations for use by CNN training 254.

At 254, CNN training may be started. CNN training 254 may ingest training data 240, for example, in batches as specified by batch size parameters 252. At 256, a gradient descent process may be applied to the ingested training data 240. As described above, gradient descent is an iterative process for performing machine learning, such as finding a minimum, or local minimum, of a function. For example, a gradient descent strategy known as RMSprop may provide improved performance with peptide binding datasets.

At 257 a loss measure may be applied to measure the loss or “cost” of processing. Examples of such loss measures may include Mean Squared Error, or cross entropy.

At 258, it may be determined whether or not quitting criteria for the gradient descent have been triggered. As gradient descent is an iterative process, criteria may be specified to determine when the iterative process should stop. At 258, if it is determined that quitting criteria for the gradient descent have not been triggered, then the process may loop back to 256, and the gradient descent process continues. At 258, if it is determined that quitting criteria for the gradient descent have been triggered (indicating that the gCNN is capable of classifying positive (real or simulated) polypeptide-MHC-I interaction data as positive and/or negative real polypeptide-MHC-I interaction data as negative), then the process may continue with 260, in which the CNN classifier model 262 may be stored as CNN classifier model 262. These stored models may include data defining the structure and coefficients that make up CNN classifier model 262. The stored models provide the capability to use CNN classifier model 262 to classify peptide bindings of input data samples, and when properly trained, provide accurate and useful results from CNN classifier model 262. At 264, CNN training ends.

At 266-280, trained convolutional neural network (CNN) classifier model 262 may be used to provide and evaluate predictions based on test data (test data can comprise one or more of positive real polypeptide-MHC-I interaction data and/or negative real polypeptide-MHC-I interaction data), so as to measure performance of the overall GAN model, as generally corresponds to 130, shown in FIG. 1. At 270, the GAN quitting criteria may be setup, for example, by setting a number of parameters 272-276 to control evaluation process 266. Examples of parameters that may be set may include accuracy of prediction parameters 272, predicting confidence parameters 274, and loss parameters 276. Accuracy of prediction parameters 272 may provide the capability to specify the accuracy of predictions to be provided by evaluation 266. For example, an accuracy threshold for predicting the real positive category can be greater than or equal to 0.9. Predicting confidence parameters 274 may provide the capability to specify the confidence levels (e.g., softmax normalization) for predictions to be provided by evaluation 266. For example, a threshold of confidence of predicting a fake or artificial category may be set to a value such as greater than or equal to 0.4, and greater than or equal to 0.6 for the real negative category. GAN quitting criteria setup processing 270 may gather training parameters 272-276, process them to be compatible with GAN prediction evaluation 266 and input the processed parameters to GAN prediction evaluation 266 or store the processed parameters in the appropriate files or locations for use by GAN prediction evaluation 266. At 266, GAN prediction evaluation may be started. GAN prediction evaluation 266 may ingest test data 268.

At 267, measurement of Area Under the Receiver Operator Characteristics (ROC) Curve (AUC) may be performed. AUC is a normalized measure of classification performance. AUC measures the likelihood that given two random points—one from the positive and one from the negative class—the classifier will rank the point from the positive class higher than the one from the negative one. In reality, it measures the performance of the ranking. AUC takes the idea that the more predicting classes that are all mixed together (in the classifier output space), the worse the classifier. ROC scans the classifier output space with a moving boundary. At each point it scans, the False Positive Rate (FPR) and True Positive Rate (TPR) are recorded (as a normalized measure). The bigger the difference between the two values, the less the points are mixed and the better they are classified. After getting all FPR and TPR pairs, they may be sorted and the ROC curve may be plotted. The AUC is the area under that curve.

At 278, it may be determined whether or not quitting criteria for the gradient descent have been triggered, generally corresponding to 140 of FIG. 1. As gradient descent is an iterative process, criteria may be specified to determine when the iterative process should stop. At 278, if it is determined that quitting criteria for evaluation process 266 have not been triggered, then the process may loop back to 220, and the training process of GAN 220-264 and the evaluation process 266 continue. Thus, when the quitting criteria is not triggered, the process will return to the GAN training (generally corresponding to returning to 110 of FIG. 1) to try produce a better generator. At 278, if it is determined that quitting criteria for evaluation process 266 have been triggered (indicating that the CNN classified positive real polypeptide-MHC-I interaction data as positive and/or negative real polypeptide-MHC-I interaction data as negative), then the process may continue with 280, in which prediction evaluation processing, and process 200 end, generally corresponding to 150 of FIG. 1.

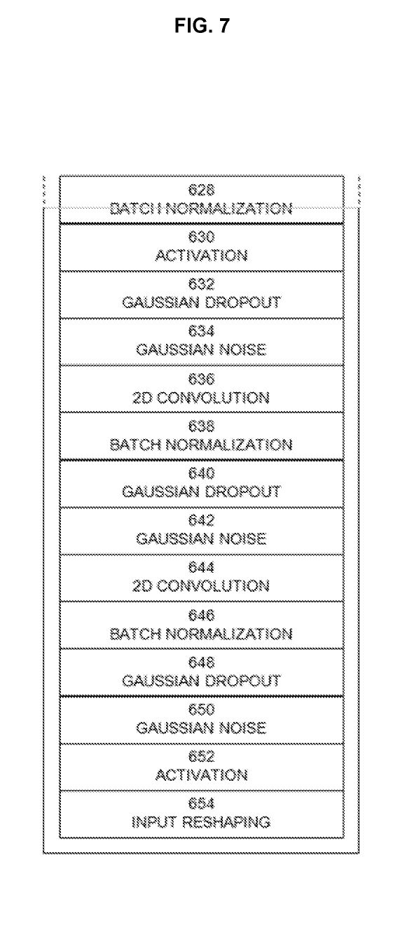

An example of an embodiment of the internal processing structure of generator 228 is shown in FIG. 6-FIG. 7. In this example, each processing block may perform the indicated type of processing, and may be performed in the order shown. It is to be noted that this is merely an example. In embodiments, the types of processing performed, as well as the order in which processing is performed, may be modified.

Turning to FIG. 6 through FIG. 7, an example processing flow for the generator 228 is described. The processing flow is only an example and is not meant to be limiting. Processing included in generator 228 may begin with dense processing 602, in which the input data inputs to a feed-forward neural layer in order to estimate the spatial variation in density of the input data. At 604, batch normalization processing may be performed. For example, normalization processing may include adjusting values measured on different scales to a common scale adjusting the entire probability distributions of the data values into alignment. Such normalization may provide improved speed of convergence, since the original (deep) neural networks is sensitive to change at layers at the beginning and the direction parameter optimizes to may be distracted by attempt to lower errors for outliers in data at the beginning. Batch normalization regularizes the gradients from these distractions and therefore is faster. At 606, activation processing may be performed. For example, activation processing may include tan h, sigmoid function, ReLU (Rectified Linear Units) or step function etc. For example, ReLU has the output 0 if the input less than 0 and the raw input otherwise. It is simpler (less computationally intense) compared to other activation functions, and therefore may provide accelerated training. At 608, input reshaping processing may be performed. For example, such processing may help to convert the shape (dimensions) of the input to a target shape that can be accepted as legitimate input in the next step. At 610, Gaussian dropout processing may be performed. Dropout is a regularization technique for reducing overfitting in neural networks based on particular training data. Dropout may be performed by deleting neural network nodes that may be causing or worsening overfitting. Gaussian dropout processing may use a Gaussian distribution to determine nodes to be deleted. Such processing may provide noise in the form of dropout, but may keep the mean and variance of inputs to their original values based on a Gaussian distribution, in order to ensure the self-normalizing property even after the dropout.

At 612, Gaussian noise processing may be performed. Gaussian noise is statistical noise having a probability density function (PDF) equal to that of the normal, or Gaussian, distribution. Gaussian noise processing may include adding noise to the data to prevent the model from learning small (often trivial) changes in the data, hence adding robustness against overfitting the model. This process may improve the prediction accuracy. At 614, two-dimensional (2D) convolutional processing may be performed. 2D convolution is an extension of 1D convolution by convolving both horizontal and vertical directions in a two-dimensional spatial domain and may provide smoothing of the data. Such processing may scan all partial inputs with multiple moving filters. Each filter may be seen as a parameter sharing neural layer that counts the occurrence of a certain feature (matching the filter parameter values) at all locations on the feature map. At 616, a second batch normalization processing may be performed. At 618, a second activation processing may be performed, at 620, a second Gaussian dropout processing may be performed, and at 622, 2D up sampling processing may be performed. Up sampling processing may transform the inputs from the original shape to a desired (mostly larger) shape. For example, resampling or interpolation may be used to do so. For example, an input may be rescaled to a desired size and the value at each point may be calculated using an interpolation such as bilinear interpolation. At 624, a second Gaussian noise processing may be performed, and at 626, a two-dimensional (2D) convolutional processing may be performed.

Continuing with FIG. 7, at 628, a third batch normalization processing may be performed, at 630, a third activation processing may be performed, at 632, a third Gaussian dropout processing may be performed, and at 634, a third Gaussian noise processing may be performed. At 636, a second two-dimensional (2D) convolutional processing may be performed, at 638, a fourth batch normalization processing may be performed. An activation processing may be performed after 638 and before 640. At 640, a fourth Gaussian dropout processing may be performed.

At 642, a fourth Gaussian noise processing may be performed, at 644, a third two-dimensional (2D) convolutional processing may be performed, and at 646, a fifth batch normalization processing may be performed. At 648, a fifth Gaussian dropout processing may be performed, at 650, a fifth Gaussian noise processing may be performed, and at 652, a fourth activation processing may be performed. This activation processing may use a sigmoid activation function, which maps an input from [−infinity,infinity] to an output of [0,1]. Typical data recognition systems may use a than activation function at the last layer. However, because the categorical nature of the present techniques, a sigmoid function may provide improved MHC binding prediction. The sigmoid function is more powerful than ReLU and may provide suitable probability output. For example, in the present classification problem, output as probability may be desirable. However, as the sigmoid function may be much slower that ReLU or tan h, it may not be desirable for performance reasons to use the sigmoid function for the previous activation layers. However, since the last dense layers are more directly related to the final output, using the sigmoid function at this activation layer may significantly improve the convergence compared to ReLU.

At 654, a second input reshaping processing may be performed to shape the output to data dimensions (that should be able to be fed to the discriminator later).

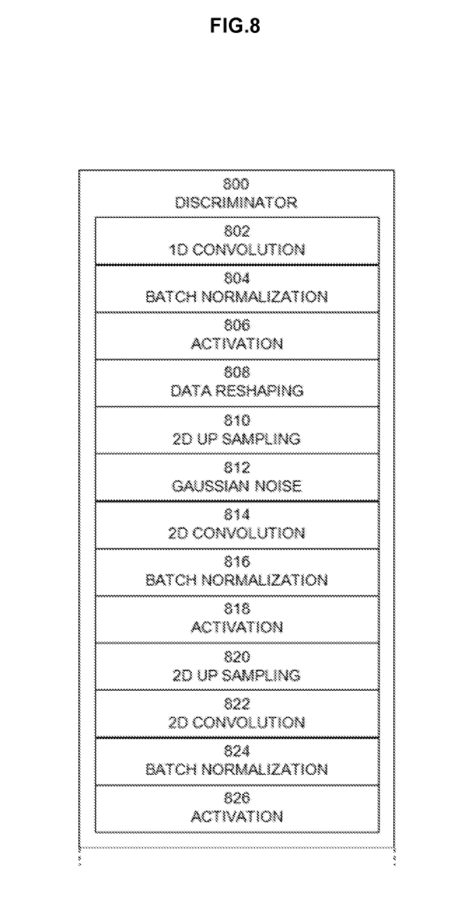

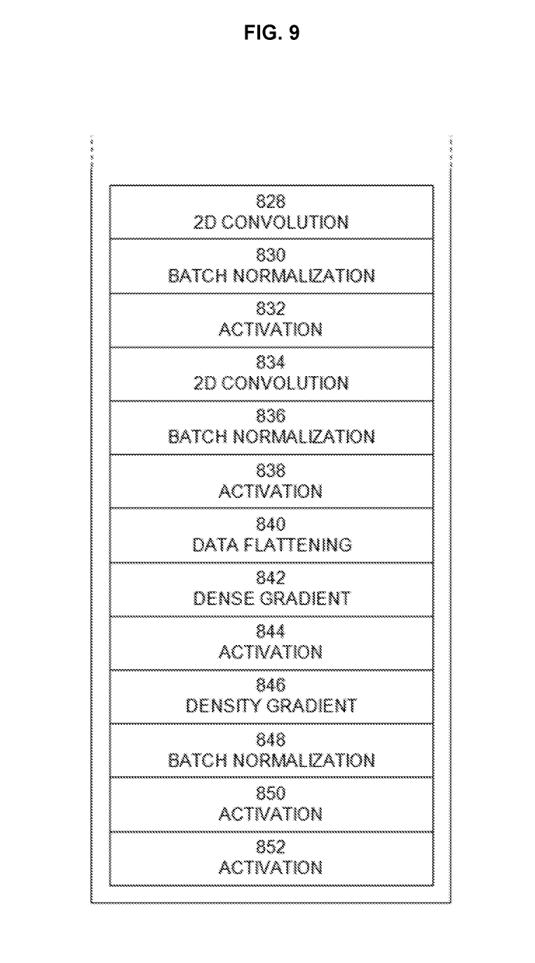

An example of an embodiment of the processing flow of discriminator 226 is shown in FIG. 8-FIG. 9. The processing flow is only an example and is not meant to be limiting. In this example, each processing block may perform the indicated type of processing, and may be performed in the order shown. It is to be noted that this is merely an example. In embodiments, the types of processing performed, as well as the order in which processing is performed, may be modified.

Turning to FIG. 8, processing included in discriminator 226 may begin with one-dimensional (1D) convolutional processing 802 which may take an input signal, apply a 1D convolutional filter on the input, and produce an output. At 804, batch normalization processing may be performed, and at 806, activation processing may be performed. For example, leaky REctifying Linear Units (RELU) processing may be used to perform the activation processing. A RELU is one type of activation function for a node or neuron in a neural network. A leaky RELU may allow a small, non-zero gradient when the node is not active (input smaller than 0). ReLU has a problem called “dying”, in which it keeps outputting 0 when the input of the activation function has a large negative bias. When this happens, the model stops learning. LeakyReLU solves this problem by providing a non-zero gradient even when it is inactive. For example, f(x)=alpha*x for x<0, f(x)=x for x>=0. At 808, input reshaping processing may be performed, and at 810, 2D up sampling processing may be performed.

Optionally, at 812, Gaussian noise processing may be performed, at 814, two-dimensional (2D) convolutional processing may be performed, at 816, a second batch normalization processing may be performed, at 818, a second activation processing may be performed, at 820, a second 2D up sampling processing may be performed, at 822, a second 2D convolutional processing may be performed, at 824, a third batch normalization processing may be performed, and at 826, third activation processing may be performed.

Continuing with FIG. 9, at 828, a third 2D convolutional processing may be performed, at 830, a fourth batch normalization processing may be performed, at 832, a fourth activation processing may be performed, at 834, a fourth 2D convolutional processing may be performed, at 836, a fifth batch normalization processing may be performed, at 838, a fifth activation processing may be performed, and at 840, a data flattening processing may be performed. For example, data flattening processing may include combining data from different tables or datasets to form a single, or a reduced number of tables or datasets. At 842, dense processing may be performed. At 844, a sixth activation processing may be performed, at 846, a second dense processing may be performed, at 848, a sixth batch normalization processing may be performed, and at 850, a seventh activation processing may be performed.

A sigmoid function may be used instead of leaky ReLU as the activation functions for the last 2 dense layers. Sigmoid is more powerful than leaky ReLU and may provide reasonable probability output (for example, in a classification problem, the output as probability is desirable). However, the sigmoid function is slower than leaky ReLU, use of the sigmoid may not be desirable for all layers. However, since the last two dense layers are more directly related to the final output, the sigmoid ay significantly improves the convergence compared to leaky ReLU. In embodiments, two dense layers (or fully connected neural network layers) 842 and 846 may be used to obtain enough complexity to transform their inputs. In particular, one dense layer may not be complex enough to transform convolutional results to discriminator output space, although it may be sufficient for use in the generator 228.

In an embodiment, methods are disclosed for using a neural network (e.g., CNN) to classify inputs based on a previous training process. The neural network can generate a prediction score and can thus classify input biological data as either successful or not successful, based upon the neural network being previously trained on a set of successful and not successful biological data including prediction scores. The prediction scores may be binding affinity scores. The neural network can be used to generate a predicted binding affinity score. The binding affinity score can numerically represent a likelihood that a single biomolecule (e.g., protein, DNA, drug, etc. . . . ) will bind to another biomolecule (e.g. protein, DNA, drug, etc. . . . ). The predicted binding affinity score can numerically represent a likelihood that a peptide (e.g., MHC) will bind to another peptide. However, machine learning techniques have thus far been unable to be brought to bear due to at least an inability to robustly make predictions when the neural network is trained on small amounts of data.

The methods and systems described address this issue by using a combination of features to more robustly make predictions. The first feature is the use of an expanded training set of biological data to train the neural network. This expanded training set is developed by training a GAN to create simulated biological data. The neural networks are then trained with this expanded training set (for example, using stochastic learning with backpropagation which is a type of machine learning algorithm that uses the gradient of a mathematical loss function to adjust the weights of the network). Unfortunately, the introduction of an expanded training set may increase false positives when classifying biological data. Accordingly, the second feature of the described methods and systems is the minimization of these false positives by performing an iterative training algorithm as needed, in which the GAN is further engaged to generate an updated simulated training set containing higher quality simulated data and the neural network is retrained with the updated training set. This combination of features provides a robust prediction model that can predict the success (e.g., binding affinity scores) of certain biological data while limiting the number of false positives.

The dataset can comprise unclassified biological data, such as unclassified protein interaction data. The unclassified biological data can comprise data regarding a protein for which no binding affinity score associated with another protein is available. The biological data can comprise a plurality of candidate protein-protein interactions, for example candidate protein-MHC-I interaction data. The CNN can generate a prediction score indicative of binding affinity and/or classify each of the candidate polypeptide-MHC-I interactions as positive or negative.

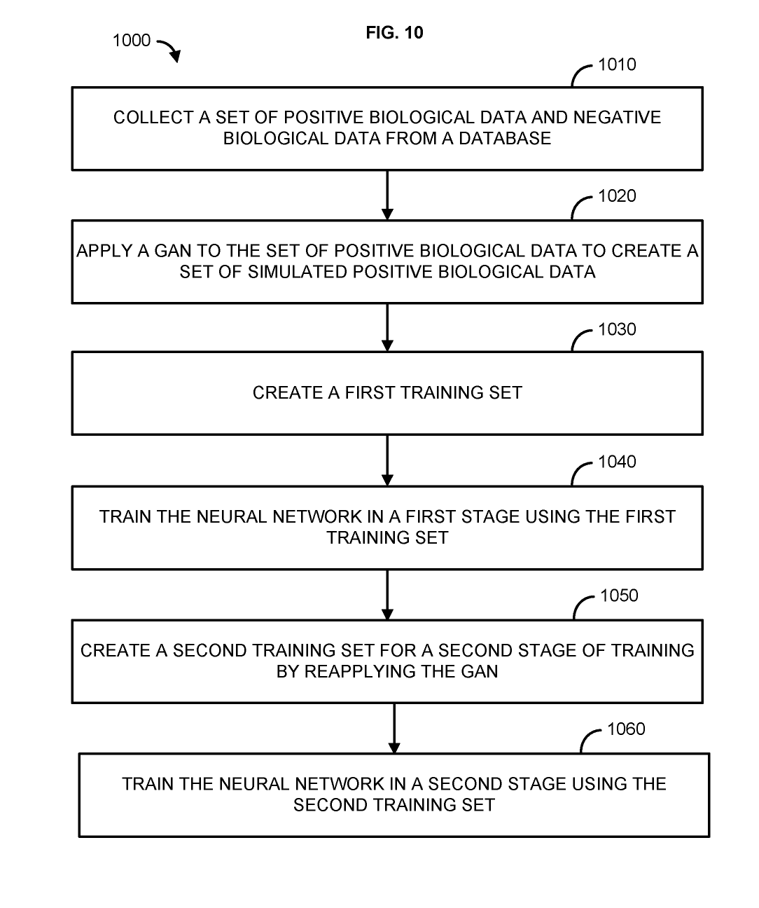

In an embodiment, shown in FIG. 10, a computer-implemented method 1000 of training a neural network for binding affinity prediction may comprise collecting a set of positive biological data and negative biological data from a database at 1010. The biological data may comprise protein-protein interaction data. The protein-protein interaction data may comprise one or more of, a sequence of a first protein, a sequence of a second protein, an identifier of the first protein, an identifier of the second protein, and/or a binding affinity score, and the like. In an embodiment, the binding affinity score may be 1, indicating successful binding (e.g., positive biological data), or −1, indicating unsuccessful binding (e.g., negative biological data).

The computer-implemented method 1000 may comprise applying a generative adversarial network (GAN) to the set of positive biological data to create a set of simulated positive biological data at 1020. Applying the GAN to the set of positive biological data to create the set of simulated positive biological data may comprise generating, by a GAN generator, increasingly accurate positive simulated biological data until a GAN discriminator classifies the positive simulated biological data as positive.

The computer-implemented method 1000 may comprise creating a first training set comprising the collected set of positive biological data, the simulated set of positive biological data, and the set of negative biological data at 1030.

The computer-implemented method 1000 may comprise training the neural network in a first stage using the first training set at 1040. Training the neural network in a first stage using the first training set may comprise presenting the positive simulated biological data, the positive biological data, and negative biological data to a convolutional neural network (CNN), until the CNN is configured to classify biological data as positive or negative.

The computer-implemented method 1000 may comprise creating a second training set for a second stage of training by reapplying the GAN to generate additional simulated positive biological data at 1050. Creating the second training set may be based on presenting the positive biological data and the negative biological data to the CNN to generate prediction scores and determining that the prediction scores are inaccurate. The prediction scores may be binding affinity scores. Inaccurate prediction scores are indicative of the CNN not being full trained which can be traced back to the GAN not being fully trained. Accordingly, one or more iterations of the GAN generator generating increasingly accurate positive simulated biological data until the GAN discriminator classifies the positive simulated biological data as positive may be performed to generate additional simulated positive biological data. The second training set may comprise the positive biological data, the simulated positive biological data, and the negative biological data.

The computer-implemented method 1000 may comprise training the neural network in a second stage using the second training set at 1060. Training the neural network in a second stage using the second training set may comprise presenting the positive biological data, the simulated positive biological data, and the negative biological data to the CNN, until the CNN is configured to classify biological data as positive or negative.

Once the CNN is fully trained, new biological data may be presented to the CNN. The new biological data may comprise protein-protein interaction data. The protein-protein interaction data may comprise one or more of a sequence of a first protein, a sequence of a second protein, an identifier of the first protein, and/or an identifier of the second protein, and the like. The CNN may analyze the new biological data and generate a prediction score (e.g., predicted binding affinity) indicative of a predicted successful or unsuccessful binding.

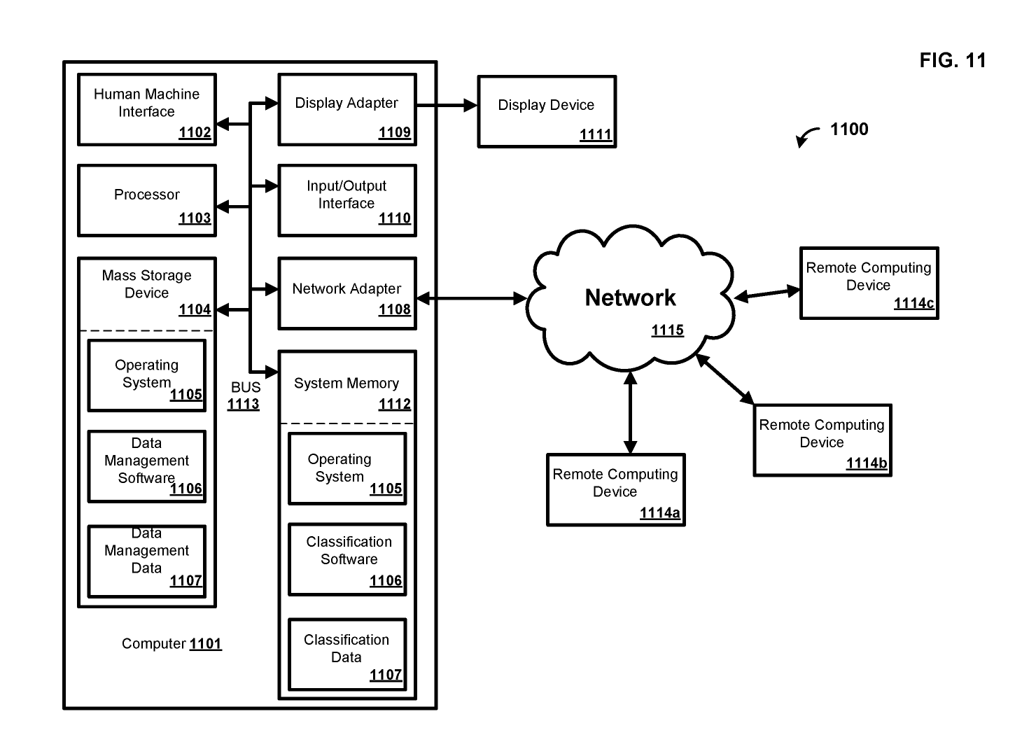

In an exemplary aspect, the methods and systems can be implemented on a computer 1101 as illustrated in FIG. 11 and described below. Similarly, the methods and systems disclosed can utilize one or more computers to perform one or more functions in one or more locations. FIG. 11 is a block diagram illustrating an exemplary operating environment for performing the disclosed methods. This exemplary operating environment is only an example of an operating environment and is not intended to suggest any limitation as to the scope of use or functionality of operating environment architecture. Neither should the operating environment be interpreted as having any dependency or requirement relating to any one or combination of components illustrated in the exemplary operating environment.

The present methods and systems can be operational with numerous other general purpose or special purpose computing system environments or configurations. Examples of well-known computing systems, environments, and/or configurations that can be suitable for use with the systems and methods comprise, but are not limited to, personal computers, server computers, laptop devices, and multiprocessor systems. Additional examples comprise set top boxes, programmable consumer electronics, network PCs, minicomputers, mainframe computers, distributed computing environments that comprise any of the above systems or devices, and the like.

The processing of the disclosed methods and systems can be performed by software components. The disclosed systems and methods can be described in the general context of computer-executable instructions, such as program modules, being executed by one or more computers or other devices. Generally, program modules comprise computer code, routines, programs, objects, components, data structures, etc. that perform particular tasks or implement particular abstract data types. The disclosed methods can also be practiced in grid-based and distributed computing environments where tasks are performed by remote processing devices that are linked through a communications network. In a distributed computing environment, program modules can be located in both local and remote computer storage media including memory storage devices.

Further, one skilled in the art will appreciate that the systems and methods disclosed herein can be implemented via a general-purpose computing device in the form of a computer 1101. The components of the computer 1101 can comprise, but are not limited to, one or more processors 1103, a system memory 1112, and a system bus 1113 that couples various system components including the one or more processors 1103 to the system memory 1112. The system can utilize parallel computing.

The system bus 1113 represents one or more of several possible types of bus structures, including a memory bus or memory controller, a peripheral bus, an accelerated graphics port, or local bus using any of a variety of bus architectures. By way of example, such architectures can comprise an Industry Standard Architecture (ISA) bus, a Micro Channel Architecture (MCA) bus, an Enhanced ISA (EISA) bus, a Video Electronics Standards Association (VESA) local bus, an Accelerated Graphics Port (AGP) bus, and a Peripheral Component Interconnects (PCI), a PCI-Express bus, a Personal Computer Memory Card Industry Association (PCMCIA), Universal Serial Bus (USB) and the like. The bus 1113, and all buses specified in this description can also be implemented over a wired or wireless network connection and each of the subsystems, including the one or more processors 1103, a mass storage device 1104, an operating system 1105, classification software 1106 (e.g., the GAN, the CNN), classification data 1107 (e.g., “real” or “simulated” data, including positive simulated polypeptide-MHC-I interaction data, positive real polypeptide-MHC-I interaction data, and/or negative real polypeptide-MHC-I interaction data), a network adapter 1108, the system memory 1112, an Input/Output Interface 1110, a display adapter 1109, a display device 1111, and a human machine interface 1102, can be contained within one or more remote computing devices 1114a,b,c at physically separate locations, connected through buses of this form, in effect implementing a fully distributed system.

The computer 1101 typically comprises a variety of computer readable media. Exemplary readable media can be any available media that is accessible by the computer 1101 and comprises, for example and not meant to be limiting, both volatile and non-volatile media, removable and non-removable media. The system memory 1112 comprises computer readable media in the form of volatile memory, such as random access memory (RAM), and/or non-volatile memory, such as read only memory (ROM). The system memory 1112 typically contains data such as the classification data 1107 and/or program modules such as the operating system 1105 and the classification software 1106 that are immediately accessible to and/or are presently operated on by the one or more processors 1103.

In another aspect, the computer 1101 can also comprise other removable/non-removable, volatile/non-volatile computer storage media. By way of example, FIG. 11 illustrates the mass storage device 1104 which can provide non-volatile storage of computer code, computer readable instructions, data structures, program modules, and other data for the computer 1101. For example and not meant to be limiting, the mass storage device 1104 can be a hard disk, a removable magnetic disk, a removable optical disk, magnetic cassettes or other magnetic storage devices, flash memory cards, CD-ROM, digital versatile disks (DVD) or other optical storage, random access memories (RAM), read only memories (ROM), electrically erasable programmable read-only memory (EEPROM), and the like.

Optionally, any number of program modules can be stored on the mass storage device 1104, including by way of example, the operating system 1105 and the classification software 1106. Each of the operating system 1105 and the classification software 1106 (or some combination thereof) can comprise elements of the programming and the classification software 1106. The classification data 1107 can also be stored on the mass storage device 1104. The classification data 1107 can be stored in any of one or more databases known in the art. Examples of such databases comprise, DB2®, Microsoft® Access, Microsoft® SQL Server, Oracle®, mySQL, PostgreSQL, and the like. The databases can be centralized or distributed across multiple systems.

In another aspect, the user can enter commands and information into the computer 1101 via an input device (not shown). Examples of such input devices comprise, but are not limited to, a keyboard, pointing device (e.g., a “mouse”), a microphone, a joystick, a scanner, tactile input devices such as gloves, and other body coverings, and the like These and other input devices can be connected to the one or more processors 1103 via the human machine interface 1102 that is coupled to the system bus 1113, but can be connected by other interface and bus structures, such as a parallel port, game port, an IEEE 1394 Port (also known as a Firewire port), a serial port, or a universal serial bus (USB).

In yet another aspect, the display device 1111 can also be connected to the system bus 1113 via an interface, such as the display adapter 1109. It is contemplated that the computer 1101 can have more than one display adapter 1109 and the computer 1101 can have more than one display device 1111. For example, the display device 1111 can be a monitor, an LCD (Liquid Crystal Display), or a projector. In addition to the display device 1111, other output peripheral devices can comprise components such as speakers (not shown) and a printer (not shown) which can be connected to the computer 1101 via the Input/Output Interface 1110. Any step and/or result of the methods can be output in any form to an output device. Such output can be any form of visual representation, including, but not limited to, textual, graphical, animation, audio, tactile, and the like. The display device 1111 and computer 1101 can be part of one device, or separate devices.

The computer 1101 can operate in a networked environment using logical connections to one or more remote computing devices 1114a,b,c. By way of example, a remote computing device can be a personal computer, portable computer, smartphone, a server, a router, a network computer, a peer device or other common network node, and so on. Logical connections between the computer 1101 and a remote computing device 1114a,b,c can be made via a network 1115, such as a local area network (LAN) and/or a general wide area network (WAN). Such network connections can be through the network adapter 1108. The network adapter 1108 can be implemented in both wired and wireless environments. Such networking environments are conventional and commonplace in dwellings, offices, enterprise-wide computer networks, intranets, and the Internet.

For purposes of illustration, application programs and other executable program components such as the operating system 1105 are illustrated herein as discrete blocks, although it is recognized that such programs and components reside at various times in different storage components of the computing device 1101, and are executed by the one or more processors 1103 of the computer. An implementation of the classification software 1106 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can comprise “computer storage media” and “communications media.” “Computer storage media” comprise volatile and non-volatile, removable and non-removable media implemented in any methods or technology for storage of information such as computer readable instructions, data structures, program modules, or other data. Exemplary computer storage media comprises, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

The methods and systems can employ Artificial Intelligence techniques such as machine learning and iterative learning. Examples of such techniques include, but are not limited to, expert systems, case based reasoning, Bayesian networks, behavior based AI, neural networks, fuzzy systems, evolutionary computation (e.g. genetic algorithms), swarm intelligence (e.g. ant algorithms), and hybrid intelligent systems (e.g. Expert inference rules generated through a neural network or production rules from statistical learning).

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the scope of the methods and systems. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### B. HLA Alleles

The disclosed systems can be trained on an unlimited number of HLA alleles. Data for peptide binding to MHC-I protein complexes encoded by HLA alleles is known in the art and available from databases including, but not limited to IEDB, AntiJen, MHCBN, SYFPEITHI, and the like.

In one embodiment, the disclosed systems and methods improve the predictability of peptide binding to MHC-I protein complexes encoded by HLA alleles: A0201, A0202, B0702, B2703, B2705, B5701, A0203, A0206, A6802, and combinations thereof. By way of example, 1028790 is the test set for A0201, A0202, A0203, A0206, A6802.

| Allele | Testset |
|---|---|
| A0201 | 1028790 |
| A0202 | 1028790 |
| B0702 | 1028928 |
| B2703 | 315174 |
| B2705 | 1029125 |
| B5701 | 1029061 |
| A0203 | 1028790 |
| A0206 | 1028790 |
| A6802 | 1028790 |

The predictability can be improved relative to existing neural systems including, but not limited to NetMHCpan, MHCflurry, sNeubula, and PSSM.

### III. Therapeutics

The disclosed systems and methods are useful for identifying peptides that bind to the MHC-I of T cells and target cells. In one embodiment, the peptides are tumor specific peptides, virus peptides, or a peptide that is displayed on the MHC-I of a target cell. The target cell can be a tumor cell, a cancer cell, or a virally infected cell. The peptides are typically displayed on antigen presenting cells, who then present the peptide antigen to CD8+ cells, for example cytotoxic T cells. Binding of the peptide antigen to the T cell activates or stimulates the T cell. Thus, one embodiment provides a vaccine, for example a cancer vaccine containing one or more peptides identified with the discloses systems and methods.

Another embodiment provides an antibody or antigen binding fragment thereof that binds to the peptide, the peptide antigen-MHC-I complex, or both.

Although specific embodiments of the present invention have been described, it will be understood by those of skill in the art that there are other embodiments that are equivalent to the described embodiments. Accordingly, it is to be understood that the invention is not to be limited by the specific illustrated embodiments, but only by the scope of the appended claims.

### EXAMPLES

### Example 1: Evaluation of Existing Predicting Models

Prediction models NetMHCpan, sNebula, MHCflurry, CNN, PSSM were evaluated. The area under ROC curve was used as the performance measurement. A value of 1 is good performance, 0 is bad performance, and 0.5 is equivalent to a random guess. Table 1 shows the models and the data used.

TABLE 1
various models for predicting peptide binding to MHC-I protein
complexes encoded by the indicated alleles

| | |
|---|---|
| NetMHCpan | Pair learning Neural Network |
| sNebula | Pair similarity cored SVM |
| MHCflurry | Ensemble of Neural Network |
| CNN | Convolutional Neural Network |
| PSSM | Position Weight Matrix |

FIG. 12 shows the evaluation data indicating that CNN trained as described herein outperforms other models at most test cases, including the current state of art, NetMHCpan. FIG. 12 shows a AUC heatmap indicating the results of applying state of the art models, and the presently described methods (“CNN ours”) to the same 15 test datasets. In FIG. 12 diagonal lines from bottom left to top right are indicative of generally higher value, the thinner the lines, the higher the value and the thicker the lines, the lower value. Diagonal lines from bottom right to top left are indicative of generally lower value, the thinner the lines, the lower the value and the thicker the lines the higher the value.

### Example 2: Problems with CNN Model

CNN training contains many random processes (e.g. Mini batch data feeding, stochastic involved in gradient by dropout, noises etc.), therefore the reproducibility of the training process can be problematic. For example, FIG. 12 shows that Vang's (“Yeeling”) AUC cannot be reproduced perfectly when implementing the exact same algorithm on the exact same data. Vang, et al., HLA class I binding prediction via convolutional neural networks, Bioinformatics, September 1; 33(17):2658-2665 (2017).

Generally speaking, a CNN is less complex than other deep learning framework like Deep Neural Network due to its parameter sharing nature, however, it is still a complex algorithm.

A standard CNN extracts features from data by a fixed size of window, but binding information on a peptide might not encode by equal lengths. In the present disclosure, as studies in biology have pointed that one type of binding mechanism happens on a scale with 7 amino acids on the peptide chain, a window size of 7 can be used and while the window size performs well, it might not be sufficient to explain other types of binding factors in all HLA binding problems.

FIG. 13A-FIG. 13C show the discrepancies between various models. FIG. 13A shows 15 test data sets from IEDB weekly-released HLA binding data. The test id is labeled by us as a unique id for all 15 test datasets. IEDB is the IEDB data release id, there may be multiple different sub dataset that relates to different HLA categories in one IEDB release. HLA is the type of HLA that binds to peptides. Length is the length of peptides binding to HLA. Test size is the number of records we have in this testing set. Training size is the number of records we have in this training set. Bind_prop is the proportion of bindings to the sum of bindings and non-bindings in the training data set, we list it here to measure the skewness of the training data. Bind_size is the number of bindings in the training data set, we use it to calculate bind_prop.

FIG. 13B-FIG. 13C show the difficulty with reproducing CNN implementation. In terms of the differences between models, there are 0 model difference in FIG. 13B-FIG. 13C. FIG. 13B-FIG. 13C show that an implementation of Adam does not match published results.

### Example 3: Bias in Data Sets

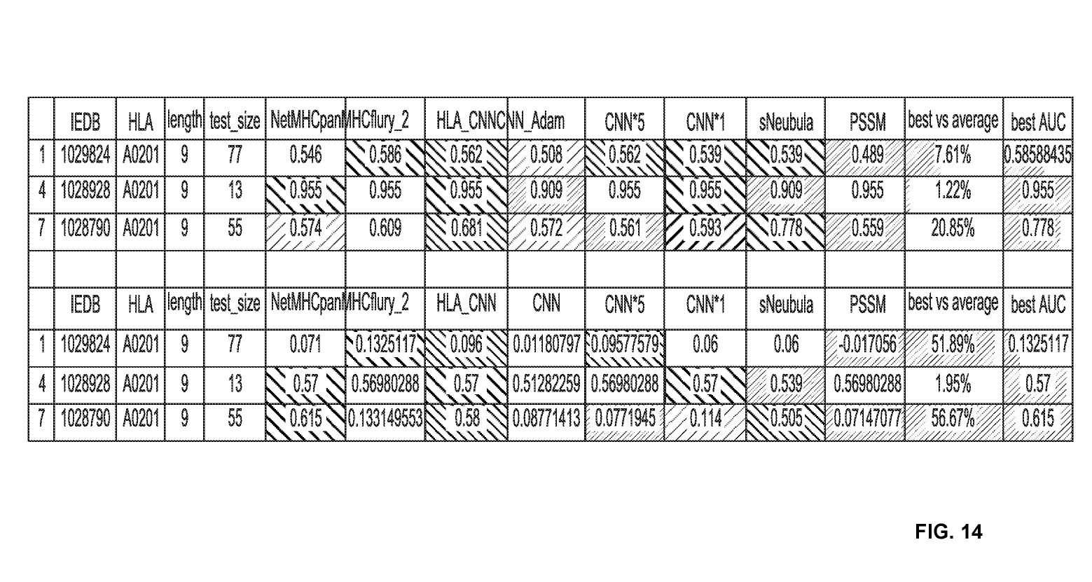

A split of train/test set was performed. The split of train/test set is a measurement designed to avoid overfitting, however, whether the measurement is effective may be dependent on data selected. Performance between the models differs significantly no matter how they are tested on the same MHC gene allele (A*02:01). This shows the AUC bias obtained by choosing a biased test set, FIG. 14. Results using the described methods on the biased train/test set are indicated in the column “CNN*1,” which shows poorer performance than that shown in FIG. 12. In FIG. 14 diagonal lines from bottom left to top right are indicative of generally higher value, the thinner the lines, the higher the value and the thicker the lines, the lower value. Diagonal lines from bottom right to top left are indicative of generally lower value, the thinner the lines, the lower the value and the thicker the lines the higher the value.

### Example 4: SRCC Bias

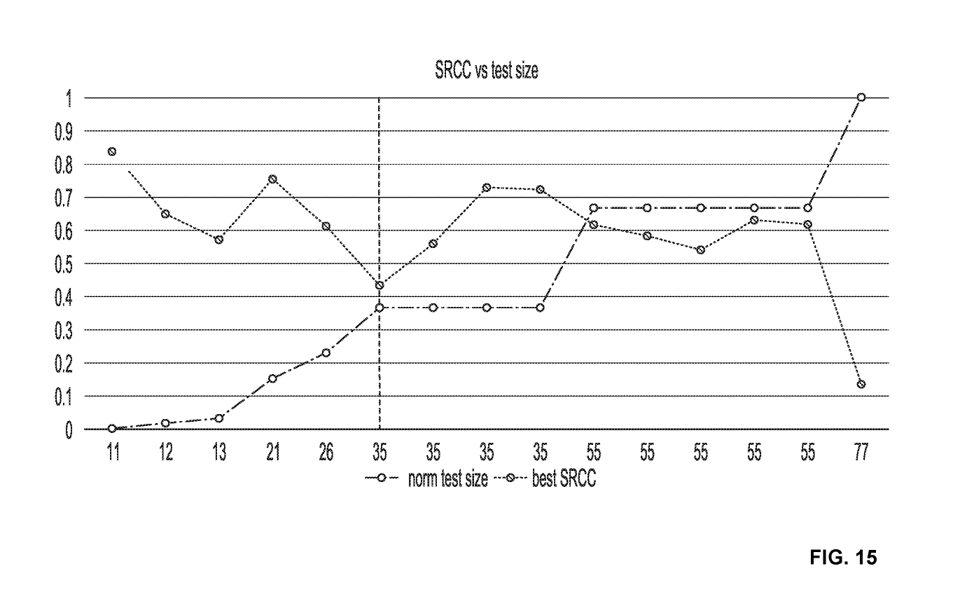

The best Spearman's rank correlation coefficient (SRCC) was selected over the 5 models tested and compared to normalized data size. FIG. 15 shows the smaller the test size, the better SRRC. SRCC measures the disorder between a prediction rank and a label rank. The bigger the test size the bigger the probability to break the ranking order.

### Example 5: Gradient Descent Comparison

A comparison between Adam and RMSprop was performed. Adam is an algorithm for first-order gradient-based optimization of stochastic objective functions, based on adaptive estimates of lower-order moments. RMSprop (for Root Mean Square Propagation) is also a method in which the learning rate is adapted for each of the parameters.

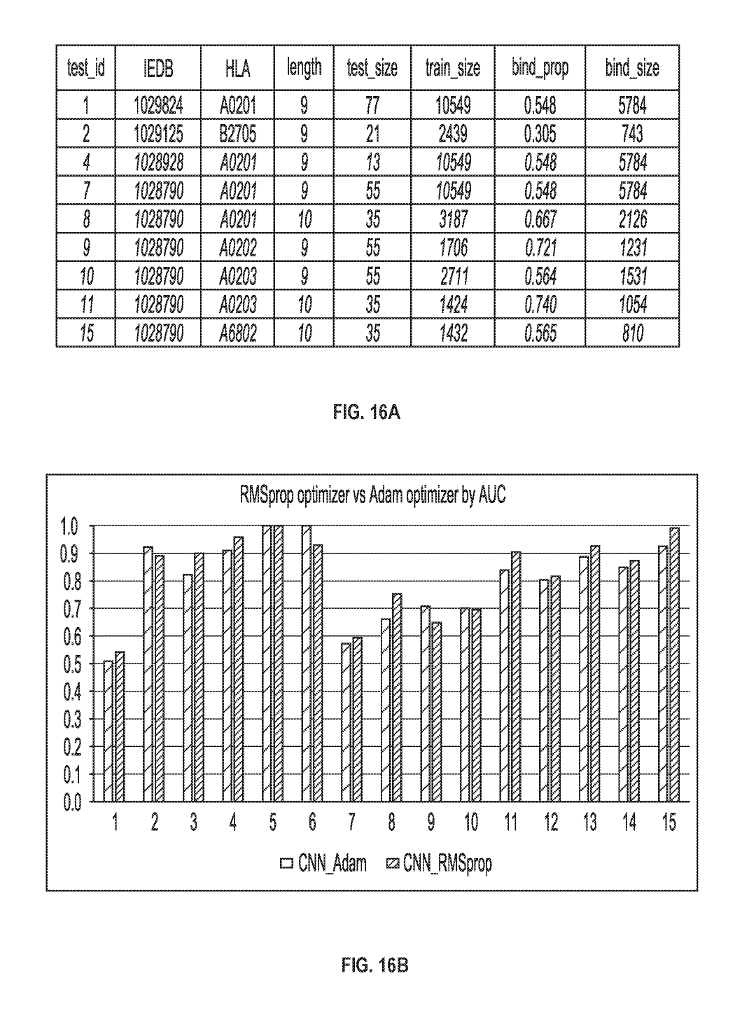

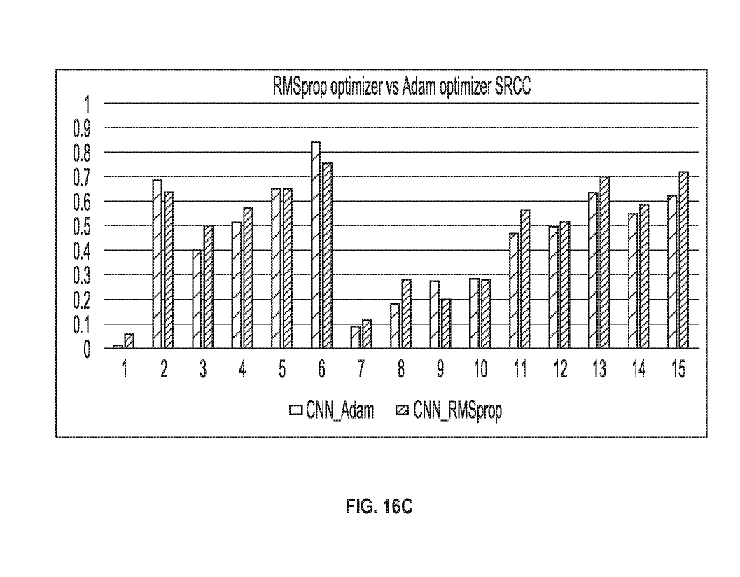

FIG. 16A-FIG. 16C show that RMSprop obtains an improvement over most of the dataset compared to Adam. Adam is a momentum based optimizer, which changes parameters aggressively in the beginning comparing to RMSprop. The improvement can relate to: 1) since the discriminator leads the entire GAN training process, if it follows the momentum and updates its parameters aggressively, then the generator end in a sub-optimal state; 2) peptide data is different than images, which tolerate fewer faults in generation. A subtle difference on the 9˜30 positions can significantly change binding results, whereas entire pixels of picture can be changed but will remain in the same category of the picture. Adam tends to explore further in the parameter zone, but it means lighter for each position in the zone; wheras RMSprop stops longer at each point and can find subtle changes on parameter pointing to a significant improvement for the final output of the discriminator, and transfer this knowledge to generator to create better simulated peptides.

### Example 5: Format of Peptide Training

Table 2 shows example MHC-I interaction data. Peptides with different binding affinity for the indicated HLA allele are shown. Peptides were designated as binding (1) or not binding (−1). Binding category was transformed from half maximal inhibitory concentration (IC₅₀). The predicted output is given in units of IC₅₀ nM. A lower number indicates a higher affinity. Peptides with IC₅₀ values<50 nM are considered high affinity, <500 nM is intermediate affinity and <5000 nM is low affinity. Most known epitopes have high or intermediated affinity. Some have low affinity. No known T-cell epitope has IC₅₀ value greater than 5000 nM.

TABLE 2
Peptides for the identified HLA allele showing
binding or no binding of the peptide to the
MHC-I protein complex encoded by the HLA allele.

| | | Binding |
|---|---|---|
| Peptide | HLA | Category |
| AAAAAAAALY (SEQ ID NO: 1) | A829:02 | 1 |
| AAAAALQAK (SEQ ID NO: 2) | A*03:01 | 1 |
| AAAAALWL (SEQ ID NO: 3) | C*16:01 | 1 |
| AAAAARAAL (SEQ ID NO: 4) | B*14:02 | −1 |
| AAAAEEEEE (SEQ ID NO: 5) | A*02:01 | −1 |
| AAAAFEAAL (SEQ ID NO: 6) | B*48:01 | 1 |
| AAAAPYAGW (SEQ ID NO: 7) | B*58:01 | 1 |
| AAAARAAAL (SEQ ID NO: 8) | B*14:02 | 1 |
| AAAATCALV (SEQ ID NO: 9) | A*02:01 | 1 |
| AAAATCALV (SEQ ID NO: 9) | A*02:02 | 1 |
| AAAATCALV (SEQ ID NO: 9) | A*02:03 | 1 |
| AAAATCALV (SEQ ID NO: 9) | A*02:06 | 1 |
| AAAATCALV (SEQ ID NO: 9) | A*68:02 | 1 |
| AAADAAAAL (SEQ ID NO: 10) | C*03:04 | 1 |
| AAADFAHAE (SEQ ID NO: 11) | B*44:03 | −1 |
| AAADPKVAF (SEQ ID NO: 12) | C*16:01 | 1 |

### Example 6: GAN Comparison

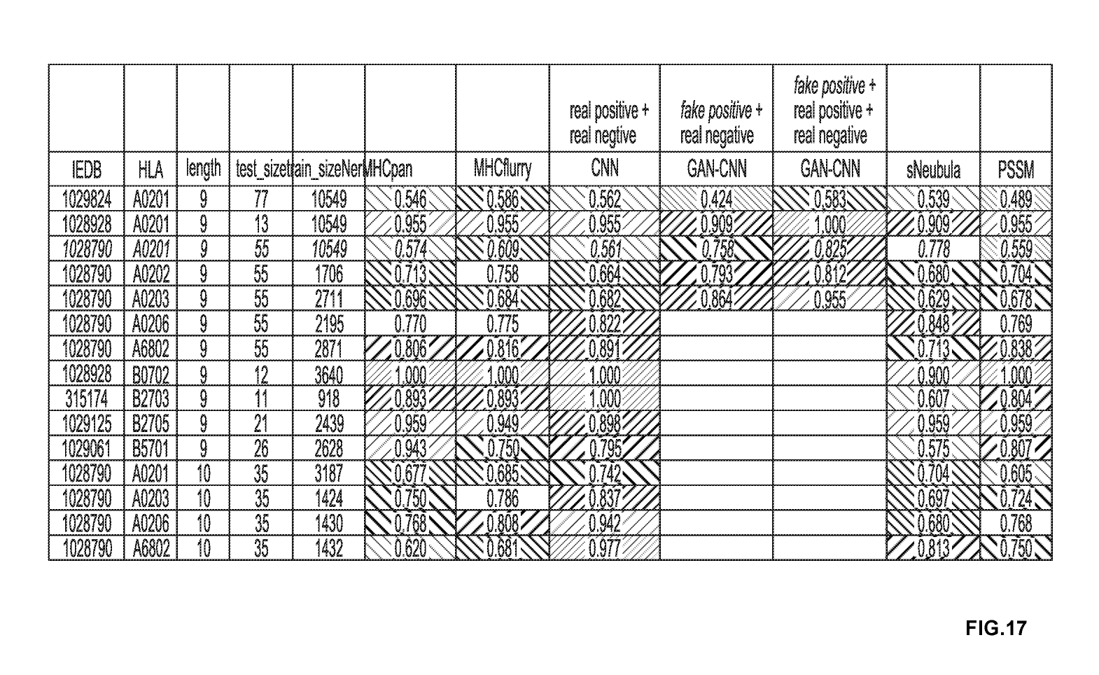

FIG. 17 shows that a mix of simulated (e.g., artificial, fake) positive data, real positive data, and real negative data results in better prediction than real positive and real negative data alone or simulated positive data and real negative data. Results from the described methods are shown in the column “CNN” and the two columns “GAN-CNN.” In FIG. 17 diagonal lines from bottom left to top right are indicative of generally higher value, the thinner the lines, the higher the value and the thicker the lines, the lower value. Diagonal lines from bottom right to top left are indicative of generally lower value, the thinner the lines, the lower the value and the thicker the lines the higher the value. GAN improves the performance of A0201 on all test sets. The use of an information extractor (e.g., CNN+skip-gram embedding) works well for peptide data as the binding information is spatially encoded. Data generated from the disclosed GAN can be seen as a way of “imputation,” which helps to make the data distributing smoother, which is easier for the model to learn. Also, the GAN's loss function makes the GAN create sharp samples rather than a blue average, which is different than classical methods such as Variational Autoencoders. Since the potential chemical binding patterns are many, average different patterns to a middle point would be sup-optimal, hence even though the GAN may overfit and face a mode-collapse issue, it will simulate patterns better.

The disclosed methods outperform state of the art systems in part due to the use of different training data. The disclosed methods outperform the use of only real positive and real negative data because the generator can enhance the frequency for some weak binding signals, which enlarges the frequency of some binding patterns, and balances the weights of different binding patterns in the training dataset, making it easier for the model to learn.

The disclosed methods outperform the use of only fake positive and real negative data because the fake positive class has a mode collapse issue, which means it cannot represent binding patterns of a whole population; similar to inputting real positive and real negative data into the model as training data but it reduces the number of training samples, resulting in the model having less data to use for learning.

In FIG. 17, the following columns are used: test id: unique for one testset, used for distinguishing testsets; IEDB: an ID for dataset on IEDB database; HLA: the allele type of the complex that binds to peptides; Length: number of amino acid of peptides; Test size: how many observations found in this testing dataset; Train_size: how many observations in this training dataset; Bind_prop: the proportion of bindings in the training dataset; Bind_size: the number of bindings in the training dataset.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; the number or type of embodiments described in the specification.

While in the foregoing specification this invention has been described in relation to certain embodiments thereof, and many details have been put forth for the purpose of illustration, it will be apparent to those skilled in the art that the invention is susceptible to additional embodiments and that certain of the details described herein can be varied considerably without departing from the basic principles of the invention.

All references cited herein are incorporated by reference in their entirety. The present invention may be embodied in other specific forms without departing from the spirit or essential attributes thereof and, accordingly, reference should be made to the appended claims, rather than to the foregoing specification, as indicating the scope of the invention.

### Example Embodiments

### Embodiment 1

A method for training a generative adversarial network (GAN), comprising: generating, by a GAN generator, increasingly accurate positive simulated polypeptide-MHC-I interaction data until a GAN discriminator classifies the positive simulated polypeptide-MHC-I interaction data as positive; presenting the positive simulated polypeptide-MHC-I interaction data, positive real polypeptide-MHC-I interaction data, and negative real polypeptide-MHC-I interaction data to a convolutional neural network (CNN), until the CNN classifies polypeptide-MHC-I interaction data as positive or negative; presenting the positive real polypeptide-MHC-I interaction data and the negative real polypeptide-MHC-I interaction data to the CNN to generate prediction scores; determining, based on the prediction scores, that the GAN is trained; and outputting the GAN and the CNN.

### Embodiment 2

The method of embodiment 1, wherein generating the increasingly accurate positive simulated polypeptide-MHC-I interaction data until the GAN discriminator classifies the positive simulated polypeptide-MHC-I interaction data as real comprises: generating, by the GAN generator according to a set of GAN parameters, a first simulated dataset comprising simulated positive polypeptide-MHC-I interactions for an MHC allele; combining the first simulated dataset with the positive real polypeptide-MHC-I interactions for the MHC allele, and the negative real polypeptide-MHC-I interactions for the MEW allele to create a GAN training dataset; determining, by a discriminator according to a decision boundary, whether a polypeptide-MHC-I interaction for the MHC allele in the GAN training dataset is simulated positive, real positive, or real negative; adjusting, based on accuracy of the determination by the discriminator, one or more of the set of GAN parameters or the decision boundary; and repeating a-d until a first stop criterion is satisfied.

### Embodiment 3

The method of embodiment 2, wherein presenting the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative comprises: generating, by the GAN generator according to the set of GAN parameters, a second simulated dataset comprising simulated positive polypeptide-MHC-I interactions for the HLA allele; combining the second simulated dataset, the positive real polypeptide-MHC-I interactions for the MEW allele, and the negative real polypeptide-MHC-I interactions for the MEW allele to create a CNN training dataset; presenting the CNN training dataset to the convolutional neural network (CNN); classifying, by the CNN according to a set of CNN parameters, a polypeptide-MHC-I interaction for the MHC allele in the CNN training dataset as positive or negative; adjusting, based on accuracy of the classification by the CNN, one or more of the set of CNN parameters; and repeating h-j until a second stop criterion is satisfied.

### Embodiment 4

The method of embodiment 3, wherein presenting the positive real polypeptide-MHC-I interaction data and the negative real polypeptide-MHC-I interaction data to the CNN to generate prediction scores comprises: classifying, by the CNN according to the set of CNN parameters, a polypeptide-MHC-I interaction for the MHC allele as positive or negative.

### Embodiment 5

The method of embodiment 4, wherein determining, based on the prediction scores, that the GAN is trained comprises determining accuracy of the classification by the CNN, wherein when (if) the accuracy of the classification satisfies a third stop criterion, outputting the GAN and the CNN.

### Embodiment 6

The method of embodiment 4 wherein determining, based on the prediction scores, that the GAN is trained comprises determining accuracy of the classification by the CNN, wherein when (if) the accuracy of the classification does not satisfy a third stop criterion, returning to step a.

### Embodiment 7

The method of embodiment 2, wherein the GAN parameters comprise one or more of allele type, allele length, generating category, model complexity, learning rate, or batch size.

### Embodiment 8

The method of embodiment 2, wherein the MHC allele is an HLA allele.

### Embodiment 9

The method of embodiment 8, wherein the HLA allele type comprises one or more of HLA-A, HLA-B, HLA-C, or a subtype thereof.

### Embodiment 10

The method of embodiment 8, wherein the HLA allele length is from about 8 to about 12 amino acids.

### Embodiment 11

The method of embodiment 8, wherein the HLA allele length is from about 9 to about 11 amino acids.

### Embodiment 12

The method of embodiment 1, further comprising: presenting a dataset to the CNN, wherein the dataset comprises a plurality of candidate polypeptide-MHC-I interactions; classifying, by the CNN, each of the plurality of candidate polypeptide-MHC-I interactions as a positive or a negative polypeptide-MHC-I interaction; and synthesizing the polypeptide from the candidate polypeptide-MHC-I interaction classified as a positive polypeptide-MHC-I interaction.

### Embodiment 13

The polypeptide produced by the method of embodiment 12.

### Embodiment 14

The method of embodiment 12, wherein the polypeptide is a tumor specific antigen.

### Embodiment 15

The method of embodiment 12, wherein the polypeptide comprises an amino acid sequence that specifically binds to an MHC-I protein encoded by a selected MHC allele.

### Embodiment 16

The method of embodiment 1, wherein the positive simulated polypeptide-MHC-I interaction data, the positive real polypeptide-MHC-I interaction data, and the negative real polypeptide-MHC-I interaction data are associated with a selected allele.

### Embodiment 17

The method of embodiment 16, wherein the selected allele is selected from a group consisting of A0201, A0202, A0203, B2703, B2705, and combinations thereof.

### Embodiment 18

The method of embodiment 1, wherein generating the increasingly accurate positive simulated polypeptide-MHC-I interaction data until the GAN discriminator classifies the positive simulated polypeptide-MHC-I interaction data as positive comprises evaluating a gradient descent expression for the GAN generator.

### Embodiment 19

The method of embodiment 1, wherein generating the increasingly accurate positive simulated polypeptide-MHC-I interaction data until the GAN discriminator classifies the positive simulated polypeptide-MHC-I interaction data as positive comprises: iteratively executing (e.g., optimizing) the GAN discriminator in order to increase a likelihood of giving a high probability to positive real polypeptide-MHC-I interaction data, a low probability to the positive simulated polypeptide-MHC-I interaction data, and a low probability to the negative real polypeptide-MHC-I interaction data; and iteratively executing (e.g., optimizing) the GAN generator in order to increase a probability of the positive simulated polypeptide-MHC-I interaction data being rated highly.

### Embodiment 20

The method of embodiment 1, wherein presenting the positive simulated polypeptide-MHC-I interaction data, the positive real polypeptide-MHC-I interaction data, and the negative real polypeptide-MHC-I interaction data to the convolutional neural network (CNN), until the CNN classifies the polypeptide-MHC-I interaction data as positive or negative comprises: performing a convolution procedure; performing a Non Linearity (ReLU) procedure; performing a Pooling or Sub Sampling procedure; and performing a Classification (Fully Connected Layer) procedure.

### Embodiment 21

The method of embodiment 1, wherein the GAN comprises a Deep Convolutional GAN (DCGAN).

### Embodiment 22

The method of embodiment 2, wherein the first stop criterion comprises evaluating a mean squared error (MSE) function.

### Embodiment 23

The method of embodiment 3, wherein the second stop criterion comprises evaluating a mean squared error (MSE) function.

### Embodiment 24

The method of embodiment 5 or 6, wherein the third stop criterion comprises evaluating an area under the curve (AUC) function.

### Embodiment 25

The method of embodiment 1, wherein the prediction score is a probability of the positive real polypeptide-MHC-I interaction data being classified as positive polypeptide-MHC-I interaction data.

### Embodiment 26

The method of embodiment 1, wherein determining, based on the prediction scores, that the GAN is trained comprises comparing one or more of the prediction scores to a threshold.

### Embodiment 27

A method for training a generative adversarial network (GAN), comprising: generating, by a GAN generator, increasingly accurate positive simulated polypeptide-MHC-I interaction data until a GAN discriminator classifies the positive simulated polypeptide-MHC-I interaction data as positive; presenting the positive simulated polypeptide-MHC-I interaction data, positive real polypeptide-MHC-I interaction data, and negative real polypeptide-MHC-I interaction data to a convolutional neural network (CNN), until the CNN classifies polypeptide-MHC-I interaction data as positive or negative; presenting the positive real polypeptide-MHC-I interaction data and the negative real polypeptide-MHC-I interaction data to the CNN to generate prediction scores; determining, based on the prediction scores, that the GAN is not trained; repeating a-c until a determination is made, based on the prediction scores, that the GAN is trained; and outputting the GAN and the CNN.

### Embodiment 28

The method of embodiment 27, wherein generating, by the GAN generator, the increasingly accurate positive simulated polypeptide-MHC-I interaction data until the GAN discriminator classifies the positive simulated polypeptide-MHC-I interaction data as positive comprises: generating, by the GAN generator according to a set of GAN parameters, a first simulated dataset comprising simulated positive polypeptide-MHC-I interactions for an MHC allele; combining the first simulated dataset with the positive real polypeptide-MHC-I interactions for the MHC allele, and the negative real polypeptide-MHC-I interactions for the MHC allele to create a GAN training dataset; determining, by a discriminator according to a decision boundary, whether a positive polypeptide-MHC-I interaction for the MHC allele in the GAN training dataset is simulated positive, real positive, or real negative; adjusting, based on accuracy of the determination by the discriminator, one or more of the set of GAN parameters or the decision boundary; and repeating g-j until a first stop criterion is satisfied.

### Embodiment 29

The method of embodiment 28, wherein presenting the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative comprises: generating, by the GAN generator according to the set of GAN parameters, a second simulated dataset comprising simulated positive polypeptide-MHC-I interactions for the MHC allele; combining the second simulated dataset, the known positive polypeptide-MHC-I interactions for the WIC allele, and the known negative polypeptide-MHC-I interactions for the WIC allele to create a CNN training dataset; presenting the CNN training dataset to the convolutional neural network (CNN); classifying, by the CNN according to a set of CNN parameters, a polypeptide-MHC-I interaction for the MHC allele in the CNN training dataset as positive or negative; adjusting, based on accuracy of the classification by the CNN, one or more of the set of CNN parameters; and repeating n-p until a second stop criterion is satisfied.

### Embodiment 30

The method of embodiment 29, wherein presenting the positive real polypeptide-MHC-I interaction data and the negative real polypeptide-MHC-I interaction data to the CNN to generate the prediction scores comprises: classifying, by the CNN according to the set of CNN parameters, a polypeptide-MHC-I interaction for the MHC allele as positive or negative.

### Embodiment 31

The method of embodiment 30, wherein determining, based on the prediction scores, that the GAN is trained comprises determining accuracy of the classification by the CNN, wherein when (if) the accuracy of the classification satisfies a third stop criterion, outputting the GAN and the CNN.

### Embodiment 32

The method of embodiment 31 wherein determining, based on the prediction scores, that the GAN is trained comprises determining accuracy of the classification by the CNN, wherein when (if) the accuracy of the classification does not satisfy a third stop criterion, returning to step a.

### Embodiment 33

The method of embodiment 28, wherein the GAN parameters comprise one or more of allele type, allele length, generating category, model complexity, learning rate, or batch size.

### Embodiment 34

The method of embodiment 33, wherein the HLA allele type comprises one or more of HLA-A, HLA-B, HLA-C, or a subtype thereof.

### Embodiment 35

The method of embodiment 33, wherein the HLA allele length is from about 8 to about 12 amino acids.

### Embodiment 36

The method of embodiment 35, wherein the HLA allele length is from about 9 to about 11 amino acids.

### Embodiment 37

The method of embodiment 27, further comprising: presenting a dataset to the CNN, wherein the dataset comprises a plurality of candidate polypeptide-MHC-I interactions; classifying, by the CNN, each of the plurality of candidate polypeptide-MHC-I interactions as a positive or a negative polypeptide-MHC-I interaction; and synthesizing the polypeptide from the candidate polypeptide-MHC-I interaction classified as a positive polypeptide-MHC-I interaction.

### Embodiment 38

The polypeptide produced by the method of embodiment 37.

### Embodiment 39

The method of embodiment 37, wherein the polypeptide is a tumor specific antigen.

### Embodiment 40

The method of embodiment 37, wherein the polypeptide comprises an amino acid sequence that specifically binds to an MHC-I protein encoded by a selected MHC allele.

### Embodiment 41

The method of embodiment 27, wherein the positive simulated polypeptide-MHC-I interaction data, the positive real polypeptide-MHC-I interaction data, and the negative real polypeptide-MHC-I interaction data are associated with a selected allele.

### Embodiment 42

The method of embodiment 41, wherein the selected allele is selected from a group consisting of A0201, A0202, A0203, B2703, B2705, and combinations thereof.

### Embodiment 43

The method of embodiment 27, wherein generating, by the GAN generator, the increasingly accurate positive simulated polypeptide-MHC-I interaction data until the GAN discriminator classifies the positive simulated polypeptide-MHC-I interaction data as positive comprises evaluating a gradient descent expression for the GAN generator.

### Embodiment 44

The method of embodiment 27, wherein generating, by the GAN generator, the increasingly accurate positive simulated polypeptide-MHC-I interaction data until the GAN discriminator classifies the positive simulated polypeptide-MHC-I interaction data as positive comprises: iteratively executing (e.g., optimizing) the GAN discriminator in order to increase a likelihood of giving a high probability to positive real polypeptide-MHC-I interaction data, a low probability to the positive simulated polypeptide-MHC-I interaction, and a low probability to the negative real polypeptide-MHC-I interaction data; and iteratively executing (e.g., optimizing) the GAN generator in order to increase a probability of the positive simulated polypeptide-MHC-I interaction data being rated highly.

### Embodiment 45

The method of embodiment 27, wherein presenting the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative comprises: performing a convolution procedure; performing a Non Linearity (ReLU) procedure; performing a Pooling or Sub Sampling procedure; and performing a Classification (Fully Connected Layer) procedure.

### Embodiment 46

The method of embodiment 27, wherein the GAN comprises a Deep Convolutional GAN (DCGAN).

### Embodiment 47

The method of embodiment 28, wherein the first stop criterion comprises evaluating a mean squared error (MSE) function.

### Embodiment 48

The method of embodiment 27, wherein the second stop criterion comprises evaluating a mean squared error (MSE) function.

### Embodiment 49

The method of embodiment 31 or 32, wherein the third stop criterion comprises evaluating an area under the curve (AUC) function.

### Embodiment 50

The method of embodiment 27, wherein the prediction score is a probability of the positive real polypeptide-MHC-I interaction data being classified as positive polypeptide-MHC-I interaction data.

### Embodiment 51

The method of embodiment 27, wherein determining, based on the prediction scores, that the GAN is trained comprises comparing one or more of the prediction scores to a threshold.

### Embodiment 52

A method for training a generative adversarial network (GAN), comprising: generating, by a GAN generator according to a set of GAN parameters, a first simulated dataset comprising simulated positive polypeptide-MHC-I interactions for a MHC allele; combining the first simulated dataset with positive real polypeptide-MHC-I interactions, and negative real polypeptide-MHC-I interactions for the MEW allele; determining, by a discriminator according to a decision boundary, whether a positive polypeptide-MHC-I interaction for the MHC allele in the GAN training dataset is positive or negative; adjusting, based on accuracy of the determination by the discriminator, one or more of the set of GAN parameters or the decision boundary; repeating a-d until a first stop criterion is satisfied; generating, by the GAN generator according to the set of GAN parameters, a second simulated dataset comprising simulated positive polypeptide-MHC-I interactions for the MHC allele; combining the second simulated dataset, the positive real polypeptide-MHC-I interactions, and the negative real polypeptide-MHC-I interactions to create a CNN training dataset; presenting the CNN training dataset to a convolutional neural network (CNN); classifying, by the CNN according to a set of CNN parameters, a polypeptide-MHC-I interaction for the MEW allele in the CNN training dataset as positive or negative; adjusting, based on accuracy of the classification by the CNN of the polypeptide-MHC-I interaction for the MHC allele in the CNN training dataset, one or more of the set of CNN parameters; repeating h-j until a second stop criterion is satisfied; presenting the CNN with the positive real polypeptide-MHC-I interaction data and the negative real polypeptide-MHC-I interaction data; classifying, by the CNN according to the set of CNN parameters, a polypeptide-MHC-I interaction for the MHC allele as positive or negative; and determining accuracy of the classification by the CNN of the polypeptide-MHC-I interaction for the MHC allele, wherein when (if) the accuracy of the classification satisfies a third stop criterion, outputting the GAN and the CNN, wherein when (if) the accuracy of the classification does not satisfy the third stop criterion, returning to step a.

### Embodiment 53

The method of embodiment 52, wherein the GAN parameters comprise one or more of allele type, allele length, generating category, model complexity, learning rate, or batch size.

### Embodiment 54

The method of embodiment 52, wherein the MHC allele is an HLA allele.

### Embodiment 55

The method of embodiment 54, wherein the HLA allele type comprises one or more of HLA-A, HLA-B, HLA-C, or a subtype thereof.

### Embodiment 56

The method of embodiment 54, wherein the HLA allele length is from about 8 to about 12 amino acids.

### Embodiment 57

The method of embodiment 54, wherein the HLA allele length is from about 9 to about 11 amino acids.

### Embodiment 58

The method of embodiment 52, further comprising: presenting a dataset to the CNN, wherein the dataset comprises a plurality of candidate polypeptide-MHC-I interactions; classifying, by the CNN, each of the plurality of candidate polypeptide-MHC-I interactions as a positive or a negative polypeptide-MHC-I interaction; and synthesizing the polypeptide from the candidate polypeptide-MHC-I interaction classified as a positive polypeptide-MHC-I interaction.

### Embodiment 59

The polypeptide produced by the method of embodiment 58.

### Embodiment 60

The method of embodiment 58, wherein the polypeptide is a tumor specific antigen.

### Embodiment 61

The method of embodiment 58, wherein the polypeptide comprises an amino acid sequence that specifically binds to an MHC-I protein encoded by a selected human leukocyte antigen (HLA) allele.

### Embodiment 62

The method of embodiment 52, wherein the positive simulated polypeptide-MHC-I interaction data, the positive real polypeptide-MHC-I interaction data, and the negative real polypeptide-MHC-I interaction data are associated with a selected allele.

### Embodiment 63

The method of embodiment 62, wherein the selected allele is selected from a group consisting of A0201, A0202, A0203, B2703, B2705, and combinations thereof.

### Embodiment 64

The method of embodiment 52, wherein repeating a-d until the first stop criterion is satisfied comprises evaluating a gradient descent expression for the GAN generator.

### Embodiment 65

The method of embodiment 52, wherein repeating a-d until the first stop criterion is satisfied comprises: iteratively executing (e.g., optimizing) the GAN discriminator in order to increase a likelihood of giving a high probability to positive real polypeptide-MHC-I interaction data, a low probability to the positive simulated polypeptide-MHC-I interaction data, and a low probability to the negative real polypeptide-MHC-I interaction data; and iteratively executing (e.g., optimizing) the GAN generator in order to increase a probability of the positive simulated polypeptide-MHC-I interaction data being rated highly.

### Embodiment 66

The method of embodiment 52, wherein presenting the CNN training dataset to the CNN comprises: performing a convolution procedure; performing a Non Linearity (ReLU) procedure; performing a Pooling or Sub Sampling procedure; and performing a Classification (Fully Connected Layer) procedure.

### Embodiment 67

The method of embodiment 52, wherein the GAN comprises a Deep Convolutional GAN (DCGAN).

### Embodiment 68

The method of embodiment 52, wherein the first stop criterion comprises evaluating a mean squared error (MSE) function.

### Embodiment 69

The method of embodiment 52, wherein the second stop criterion comprises evaluating a mean squared error (MSE) function.

### Embodiment 70

The method of embodiment 52, wherein the third stop criterion comprises evaluating an area under the curve (AUC) function.

### Embodiment 71

A method comprising: training a convolutional neural network (CNN) according to the method of embodiment 1; presenting a dataset to the CNN, wherein the dataset comprises a plurality of candidate polypeptide-MHC-I interactions; classifying, by the CNN, each of the plurality of candidate polypeptide-MHC-I interactions as a positive or a negative polypeptide-MHC-I interaction; and synthesizing a polypeptide associated with a candidate polypeptide-MHC-I interaction classified as a positive polypeptide-MHC-I interaction.

### Embodiment 72

The method of embodiment 71, wherein the CNN is trained based on one or more GAN parameters comprising one or more of allele type, allele length, generating category, model complexity, learning rate, or batch size.

### Embodiment 73

The method of embodiment 72, wherein the allele type is an HLA allele type.

### Embodiment 74

The method of embodiment 73, wherein the HLA allele type comprises one or more of HLA-A, HLA-B, HLA-C, or a subtype thereof.

### Embodiment 75

The method of embodiment 73, wherein the HLA allele length is from about 8 to about 12 amino acids.

### Embodiment 76

The method of embodiment 73, wherein the HLA allele length is from about 9 to about 11 amino acids.

### Embodiment 77

The polypeptide produced by the method of embodiment 71.

### Embodiment 78

The method of embodiment 71, wherein the polypeptide is a tumor specific antigen.

### Embodiment 79

The method of embodiment 71, wherein the polypeptide comprises an amino acid sequence that specifically binds to an MHC-I protein encoded by a selected human leukocyte antigen (HLA) allele.

### Embodiment 80

The method of embodiment 71, wherein the positive simulated polypeptide-MHC-I interaction data, the positive real polypeptide-MHC-I interaction data, and the negative real polypeptide-MHC-I interaction data are associated with a selected allele.

### Embodiment 81

The method of embodiment 80, wherein the selected allele is selected from a group consisting of A0201, A0202, A0203, B2703, B2705, and combinations thereof.

### Embodiment 82

The method of embodiment 71, wherein the GAN comprises a Deep Convolutional GAN (DCGAN).

### Embodiment 83

An apparatus for training a generative adversarial network (GAN), comprising: one or more processors; and memory storing processor executable instructions that, when executed by the one or more processors, cause the apparatus to: generate increasingly accurate positive simulated polypeptide-MHC-I interaction data until a GAN discriminator classifies the positive simulated polypeptide-MHC-I interaction data as positive; present the positive simulated polypeptide-MHC-I interaction data, positive real polypeptide-MHC-I interaction data, and negative real polypeptide-MHC-I interaction data to a convolutional neural network (CNN), until the CNN classifies polypeptide-MHC-I interaction data as positive or negative; present the positive real polypeptide-MHC-I interaction data and the negative real polypeptide-MHC-I interaction data to the CNN to generate prediction scores; determine, based on the prediction scores, that the GAN is trained; and output the GAN and the CNN.

### Embodiment 84

The apparatus of embodiment 83, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to: generate, according to a set of GAN parameters, a first simulated dataset comprising simulated positive polypeptide-MHC-I interactions for an MHC allele; combine the first simulated dataset the positive real polypeptide-MHC-I interactions for the MHC allele, and the negative real polypeptide-MHC-I interactions for the WIC allele to create a GAN training dataset; receive information from a discriminator, wherein the discriminator is configured to determine, according to a decision boundary, whether a positive polypeptide-MHC-I interaction for the MHC allele in the GAN training dataset is positive or negative; adjust, based on accuracy of the information from the discriminator, one or more of the set of GAN parameters or the decision boundary; and repeat a-d until a first stop criterion is satisfied.

### Embodiment 85

The apparatus of embodiment 84, wherein the processor executable instructions that, when executed by the one or more processors, cause the apparatus to present the positive simulated polypeptide-MHC-I interaction data, the positive real polypeptide-MHC-I interaction data, and the negative real polypeptide-MHC-I interaction data to a convolutional neural network (CNN), until the CNN classifies polypeptide-MHC-I interaction data as positive or negative further comprise processor executable instructions that, when executed by the one or more processors, cause the apparatus to: generate, according to the set of GAN parameters, a second simulated dataset comprising simulated positive polypeptide-MHC-I interactions for the WIC allele; combine the second simulated dataset, the positive real polypeptide-MHC-I interaction data for the MHC allele, and the negative real polypeptide-MHC-I interaction data for the MHC allele to create a CNN training dataset; present the CNN training dataset to a convolutional neural network (CNN); receive training information from the CNN, wherein the CNN is configured to determine the training information by classifying, according to a set of CNN parameters, a polypeptide-MHC-I interaction for the MHC allele in the CNN training dataset as positive or negative; adjust, based on accuracy of training information, one or more of the set of CNN parameters; and repeat h-j until a second stop criterion is satisfied.

### Embodiment 86

The apparatus of embodiment 85, wherein the processor executable instructions that, when executed by the one or more processors, cause the apparatus to present the positive real polypeptide-MHC-I interaction data and the negative real polypeptide-MHC-I interaction data to the CNN to generate prediction scores further comprise processor executable instructions that, when executed by the one or more processors, cause the apparatus to: classify, according to the set of CNN parameters, a polypeptide-MHC-I interaction for the MHC allele as positive or negative.

### Embodiment 87

The apparatus of embodiment 86, wherein the processor executable instructions that, when executed by the one or more processors, cause the apparatus to determine, based on the prediction scores, that the GAN is trained further comprise processor executable instructions that, when executed by the one or more processors, cause the apparatus to determine accuracy of the classification of the polypeptide-MHC-I interaction for the MHC allele as positive or negative, and when (if) the accuracy of the classification satisfies a third stop criterion, output the GAN and the CNN.

### Embodiment 88

The apparatus of embodiment 86, wherein the processor executable instructions that, when executed by the one or more processors, cause the apparatus to determine, based on the prediction scores, that the GAN is trained further comprise processor executable instructions that, when executed by the one or more processors, cause the apparatus to determine accuracy of the classification of the polypeptide-MHC-I interaction for the MHC allele as positive or negative, and when (if) the accuracy of the classification does not satisfy a third stop criterion, return to step a.

### Embodiment 89

The apparatus of embodiment 84, wherein the GAN parameters comprise one or more of allele type, allele length, generating category, model complexity, learning rate, or batch size.

### Embodiment 90

The apparatus of embodiment 89, wherein the HLA allele type comprises one or more of HLA-A, HLA-B, HLA-C, or a subtype thereof.

### Embodiment 91

The apparatus of embodiment 89, wherein the HLA allele length is from about 8 to about 12 amino acids.

### Embodiment 92

The apparatus of embodiment 89, wherein the HLA allele length is from about 9 to about 11 amino acids.

### Embodiment 93

The apparatus of embodiment 83, wherein the processor executable instructions, when executed by the one or more processors, further cause the apparatus to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesize the polypeptide from the candidate polypeptide-MHC-I interaction that the CNN classifies as a positive polypeptide-MHC-I interaction.

### Embodiment 94

The polypeptide produced by the apparatus of embodiment 93.

### Embodiment 95

The apparatus of embodiment 93, wherein the polypeptide is a tumor specific antigen.

### Embodiment 96

The apparatus of embodiment 93, wherein the polypeptide comprises an amino acid sequence that specifically binds to an MHC-I protein encoded by a selected human leukocyte antigen (HLA) allele.

### Embodiment 97

The apparatus of embodiment 83, wherein the positive simulated polypeptide-MHC-I interaction data, the positive real polypeptide-MHC-I interaction data, and the negative real polypeptide-MHC-I interaction data are associated with a selected allele.

### Embodiment 98

The apparatus of embodiment 97, wherein the selected allele is selected from a group consisting of A0201, A0202, A0203, B2703, B2705, and combinations thereof.

### Embodiment 99

The apparatus of embodiment 83, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to evaluate a gradient descent expression for the GAN generator.

### Embodiment 100

The apparatus of embodiment 83, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to: iteratively execute (e.g., optimize) the GAN discriminator in order to increase a likelihood of giving a high probability to positive real polypeptide-MHC-I interaction data, a low probability to the positive simulated polypeptide-MHC-I interaction data, and a low probability to the negative simulated polypeptide-MHC-I interaction data; and iteratively execute (e.g., optimize) the GAN generator in order to increase a probability of the positive simulated polypeptide-MHC-I interaction data being rated highly.

### Embodiment 101

The apparatus of embodiment 83, wherein the processor executable instructions that, when executed by the one or more processors, cause the apparatus to present the positive simulated polypeptide-MHC-I interaction data, the positive real polypeptide-MHC-I interaction data, and the negative real polypeptide-MHC-I interaction data to the convolutional neural network (CNN), until the CNN classifies the polypeptide-MHC-I interaction data as positive or negative real further comprise processor executable instructions that, when executed by the one or more processors, cause the apparatus to: perform a convolution procedure; perform a Non Linearity (ReLU) procedure; perform a Pooling or Sub Sampling procedure; and perform a Classification (Fully Connected Layer) procedure.

### Embodiment 102

The apparatus of embodiment 83, wherein the GAN comprises a Deep Convolutional GAN (DCGAN).

### Embodiment 103

The apparatus of embodiment 84, wherein the first stop criterion comprises an evaluation of a mean squared error (MSE) function.

### Embodiment 104

The apparatus of embodiment 85, wherein the second stop criterion comprises an evaluation of a mean squared error (MSE) function.

### Embodiment 105

The apparatus of embodiment 87 or 88, wherein the third stop criterion comprises an evaluation of an area under the curve (AUC) function.

### Embodiment 106

The apparatus of embodiment 83, wherein the prediction score is a probability of the positive real polypeptide-MHC-I interaction data being classified as positive polypeptide-MHC-I interaction data.

### Embodiment 107

The apparatus of embodiment 83, wherein the processor executable instructions that, when executed by the one or more processors, cause the apparatus to determine, based on the prediction scores, that the GAN is trained further comprise processor executable instructions that, when executed by the one or more processors, cause the apparatus to compare one or more of the prediction scores to a threshold.

### Embodiment 108

An apparatus for training a generative adversarial network (GAN), comprising:

one or more processors; and
memory storing processor executable instructions that, when executed by the one or more processors, cause the apparatus to: generate increasingly accurate positive simulated polypeptide-MHC-I interaction data until a GAN discriminator classifies the positive simulated polypeptide-MHC-I interaction data as positive; present the positive simulated polypeptide-MHC-I interaction data, positive real polypeptide-MHC-I interaction data, and negative real polypeptide-MHC-I interaction data to a convolutional neural network (CNN), until the CNN classifies polypeptide-MHC-I interaction data as positive or negative; present the positive real polypeptide-MHC-I interaction data and the negative real polypeptide-MHC-I interaction data to the CNN to generate prediction scores; determine, based on the prediction scores, that the GAN is not trained; repeat a-c until a determination is made, based on the prediction scores, that the GAN is trained; and output the GAN and the CNN.

### Embodiment 109

The apparatus of embodiment 108, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to: generate, according to a set of GAN parameters, a first simulated dataset comprising simulated positive polypeptide-MHC-I interactions for an MHC allele; combine the first simulated dataset with the positive real polypeptide-MHC-I interactions for the MHC allele, and the positive real polypeptide-MHC-I interactions for the MHC allele to create a GAN training dataset; receive information from a discriminator, wherein the discriminator is configured to determine, whether a positive polypeptide-MHC-I interaction for the MHC allele in the GAN training dataset is positive or negative; adjust, based on accuracy of the information from the discriminator, one or more of the set of GAN parameters or the decision boundary; and repeat i-j until a first stop criterion is satisfied.

### Embodiment 110

The apparatus of embodiment 109, wherein the processor executable instructions that, when executed by the one or more processors, cause the apparatus to present the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative further comprise processor executable instructions that, when executed by the one or more processors, cause the apparatus to: generate, according to the set of GAN parameters, a second simulated dataset comprising simulated positive polypeptide-MHC-I interactions for the WIC allele; combine the second simulated dataset, the positive real polypeptide-MHC-I interaction data, and the negative real polypeptide-MHC-I interaction data to create a CNN training dataset; present the CNN training dataset to the convolutional neural network (CNN); receive information from the CNN, wherein the CNN is configured to determine the information by classifying, according to a set of CNN parameters, a polypeptide-MHC-I interaction for the MHC allele in the CNN training dataset as positive or negative; adjust, based on accuracy of the information from the CNN, one or more of the set of CNN parameters; and repeat n-p until a second stop criterion is satisfied.

### Embodiment 111

The apparatus of embodiment 110, wherein the processor executable instructions that, when executed by the one or more processors, cause the apparatus to present the positive real polypeptide-MHC-I interaction data and the negative real polypeptide-MHC-I interaction data to the CNN to generate the prediction scores further comprise processor executable instructions that, when executed by the one or more processors, cause the apparatus to: present the CNN with the positive real polypeptide-MHC-I interaction data and the negative real polypeptide-MHC-I interaction data, wherein the CNN is further configured to classify, according to the set of CNN parameters, a polypeptide-MHC-I interaction for the MHC allele as positive or negative.

### Embodiment 112

The apparatus of embodiment 111, wherein the processor executable instructions that, when executed by the one or more processors, cause the apparatus to determine, based on the prediction scores, that the GAN is trained further comprise processor executable instructions that, when executed by the one or more processors, cause the apparatus to: determine accuracy of the classification by the CNN; determine that the accuracy of the classification satisfies a third stop criterion; and in response to determining that the accuracy of the classification satisfies the third stop criterion, output the GAN and the CNN.

### Embodiment 113

The apparatus of embodiment 112, wherein the processor executable instructions that, when executed by the one or more processors, cause the apparatus to determine, based on the prediction scores, that the GAN is trained further comprise processor executable instructions that, when executed by the one or more processors, cause the apparatus to: determine accuracy of the classification by the CNN; determine that the accuracy of the classification does not satisfy a third stop criterion; and in response to determining that the accuracy of the classification does not satisfy the third stop criterion, returning to step a.

### Embodiment 114

The apparatus of embodiment 109, wherein the GAN parameters comprise one or more of allele type, allele length, generating category, model complexity, learning rate, or batch size.

### Embodiment 115

The apparatus of embodiment 109, wherein the MHC allele is an HLA allele.

### Embodiment 116

The apparatus of embodiment 115, wherein the HLA allele type comprises one or more of HLA-A, HLA-B, HLA-C, or a subtype thereof.

### Embodiment 117

The apparatus of embodiment 115, wherein the HLA allele length is from about 8 to about 12 amino acids.

### Embodiment 118

The apparatus of embodiment 115, wherein the HLA allele length is from about 9 to about 11 amino acids.

### Embodiment 119

The apparatus of embodiment 108, wherein the processor executable instructions, when executed by the one or more processors, further cause the apparatus to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesize the polypeptide from the candidate polypeptide-MHC-I interaction classified by the CNN as a positive polypeptide-MHC-I interaction.

### Embodiment 120

The polypeptide produced by the apparatus of embodiment 119.

### Embodiment 121

The apparatus of embodiment 119, wherein the polypeptide is a tumor specific antigen.

### Embodiment 122

The apparatus of embodiment 119, wherein the polypeptide comprises an amino acid sequence that specifically binds to an MHC-I protein encoded by a selected human leukocyte antigen (HLA) allele.

### Embodiment 123

The apparatus of embodiment 108, wherein the positive simulated polypeptide-MHC-I interaction data, the positive real polypeptide-MHC-I interaction data, and the negative real polypeptide-MHC-I interaction data are associated with a selected allele.

### Embodiment 124

The apparatus of embodiment 123, wherein the selected allele is selected from a group consisting of A0201, A0202, A0203, B2703, B2705, and combinations thereof.

### Embodiment 125

The apparatus of embodiment 108, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to evaluate a gradient descent expression for the GAN generator.

### Embodiment 126

The apparatus of embodiment 108, wherein the processor executable instructions that, when executed by the one or more processors, cause the apparatus to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the apparatus to: iteratively execute (e.g., optimize) the GAN discriminator in order to increase a likelihood of giving a high probability to positive real polypeptide-MHC-I interaction data, a low probability to the positive simulated polypeptide-MHC-I interaction data, and a low probability to the negative simulated polypeptide-MHC-I interaction data; and iteratively execute (e.g., optimize) the GAN generator in order to increase a probability of the positive simulated polypeptide-MHC-I interaction data being rated highly.

### Embodiment 127

The apparatus of embodiment 108, wherein the processor executable instructions that, when executed by the one or more processors, cause the apparatus to present the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative further comprise processor executable instructions that, when executed by the one or more processors, cause the apparatus to: perform a convolution procedure; perform a Non Linearity (ReLU) procedure; perform a Pooling or Sub Sampling procedure; and perform a Classification (Fully Connected Layer) procedure.

### Embodiment 128

The apparatus of embodiment 108, wherein the GAN comprises a Deep Convolutional GAN (DCGAN).

### Embodiment 129

The apparatus of embodiment 109, wherein the first stop criterion comprises an evaluation of a mean squared error (MSE) function.

### Embodiment 130

The apparatus of embodiment 108, wherein the second stop criterion comprises an evaluation of a mean squared error (MSE) function.

### Embodiment 131

The apparatus of embodiment 112 or 113, wherein the third stop criterion comprises an evaluation of an area under the curve (AUC) function.

### Embodiment 132

The apparatus of embodiment 108, wherein the prediction score is a probability of the positive real polypeptide-MHC-I interaction data being classified as positive polypeptide-MHC-I interaction data.

### Embodiment 133

The apparatus of embodiment 108, wherein the processor executable instructions that, when executed by the one or more processors, cause the apparatus to determine, based on the prediction scores, that the GAN is trained further comprise processor executable instructions that, when executed by the one or more processors, cause the apparatus to compare one or more of the prediction scores to a threshold.

### Embodiment 134

An apparatus for training a generative adversarial network (GAN), comprising: one or more processors; and memory storing processor executable instructions that, when executed by the one or more processors, cause the apparatus to: generate, according to a set of GAN parameters, a first simulated dataset comprising simulated positive polypeptide-MHC-I interactions for an MHC allele; combine the first simulated dataset with positive real polypeptide-MHC-I interactions for the WIC allele and negative real polypeptide-MHC-I interactions for the WIC allele to create a GAN training dataset; receive information from a discriminator, wherein the discriminator is configured to determine, according to a decision boundary, whether a positive polypeptide-MHC-I interaction for the MHC allele in the GAN training dataset is positive or negative; adjust, based on accuracy of the information from the discriminator, one or more of the set of GAN parameters or the decision boundary; repeat a-d until a first stop criterion is satisfied; generate, by the GAN generator according to the set of GAN parameters, a second simulated dataset comprising simulated positive polypeptide-MHC-I interactions for the WIC allele; combine the second simulated dataset, the positive real polypeptide-MHC-I interaction data, and the negative real polypeptide-MHC-I interaction data for the MHC allele to create a CNN training dataset; present the CNN training dataset to a convolutional neural network (CNN); receive training information from the CNN, wherein the CNN is configured to determine the training information by classifying, according to a set of CNN parameters, a polypeptide-MHC-I interaction for the MHC allele in the CNN training dataset as positive or negative; adjust, based on accuracy of the training information, one or more of the set of CNN parameters; repeat h-j until a second stop criterion is satisfied; present the CNN with the positive real polypeptide-MHC-I interactions for the WIC allele, and the negative real polypeptide-MHC-I interactions for the MHC allele; receive training information from the CNN, wherein the CNN is configured to determine the training information by classifying, according to the set of CNN parameters, a polypeptide-MHC-I interaction for the MHC allele as positive or negative; and determine accuracy of the training information, wherein when (if) the accuracy of the training information satisfies a third stop criterion, outputting the GAN and the CNN, wherein when (if) the accuracy of the training information does not satisfy the third stop criterion, returning to step a.

### Embodiment 135

The apparatus of embodiment 134, wherein the GAN parameters comprise one or more of allele type, allele length, generating category, model complexity, learning rate, or batch size.

### Embodiment 136

The apparatus of embodiment 134, wherein the MHC allele is an HLA allele.

### Embodiment 137

The apparatus of embodiment 136, wherein the HLA allele type comprises one or more of HLA-A, HLA-B, HLA-C, or a subtype thereof.

### Embodiment 138

The apparatus of embodiment 136, wherein the HLA allele length is from about 8 to about 12 amino acids.

### Embodiment 139

The apparatus of embodiment 136, wherein the HLA allele length is from about 9 to about 11 amino acids.

### Embodiment 140

The apparatus of embodiment 134, wherein the processor executable instructions, when executed by the one or more processors, further cause the apparatus to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesizing the polypeptide from the candidate polypeptide-MHC-I interaction classified by the CNN as a positive polypeptide-MHC-I interaction.

### Embodiment 141

The polypeptide produced by the apparatus of embodiment 140.

### Embodiment 142

The apparatus of embodiment 140, wherein the polypeptide is a tumor specific antigen.

### Embodiment 143

The apparatus of embodiment 140, wherein the polypeptide comprises an amino acid sequence that specifically binds to an MHC-I protein encoded by a MHC allele.

### Embodiment 144

The apparatus of embodiment 134, wherein the positive simulated polypeptide-MHC-I interaction data, the positive real polypeptide-MHC-I interaction data, and the negative real polypeptide-MHC-I interaction data are associated with a selected allele.

### Embodiment 145

The apparatus of embodiment 144, wherein the selected allele is selected from a group consisting of A0201, A0202, A0203, B2703, B2705, and combinations thereof.

### Embodiment 146

The apparatus of embodiment 134, wherein the processor executable instructions that, when executed by the one or more processors, cause the apparatus to repeat a-d until the first stop criterion is satisfied further comprise processor executable instructions that, when executed by the one or more processors, cause the apparatus to evaluate a gradient descent expression for the GAN generator.

### Embodiment 147

The apparatus of embodiment 134, wherein the processor executable instructions that, when executed by the one or more processors, cause the apparatus to repeat a-d until the first stop criterion is satisfied further comprise processor executable instructions that, when executed by the one or more processors, cause the apparatus to: iteratively execute (e.g., optimize) the GAN discriminator in order to increase a likelihood of giving a high probability to positive real polypeptide-MHC-I interaction data, a low probability to the positive simulated polypeptide-MHC-I interaction data, and a low probability to the negative simulated polypeptide-MHC-I interaction data; and iteratively execute (e.g., optimize) the GAN generator in order to increase a probability of the positive simulated polypeptide-MHC-I interaction data being rated highly.

### Embodiment 148

The apparatus of embodiment 134, wherein the processor executable instructions that, when executed by the one or more processors, cause the apparatus to present the CNN training dataset to the CNN further comprise processor executable instructions that, when executed by the one or more processors, cause the apparatus to: perform a convolution procedure; perform a Non Linearity (ReLU) procedure; perform a Pooling or Sub Sampling procedure; and perform a Classification (Fully Connected Layer) procedure.

### Embodiment 149

The apparatus of embodiment 134, wherein the GAN comprises a Deep Convolutional GAN (DCGAN).

### Embodiment 150

The apparatus of embodiment 134, wherein the first stop criterion comprises an evaluation of a mean squared error (MSE) function.

### Embodiment 151

The apparatus of embodiment 134, wherein the second stop criterion comprises an evaluation of a mean squared error (MSE) function.

### Embodiment 152

The apparatus of embodiment 134, wherein the third stop criterion comprises an evaluation of an area under the curve (AUC) function.

### Embodiment 153

An apparatus comprising: one or more processors; and memory storing processor executable instructions that, when executed by the one or more processors, cause the apparatus to: train a convolutional neural network (CNN) by the same means as the apparatus of embodiment 83; present a dataset to the CNN, wherein the dataset comprises a plurality of candidate polypeptide-MHC-I interactions, wherein the CNN is configured to classify each of the plurality of candidate polypeptide-MHC-I interactions as a positive or a negative polypeptide-MHC-I interaction; and synthesize a polypeptide associated with a candidate polypeptide-MHC-I interaction classified by the CNN as a positive polypeptide-MHC-I interaction.

### Embodiment 154

The apparatus of embodiment 153, wherein the CNN is trained based on one or more GAN parameters comprising one or more of allele type, allele length, generating category, model complexity, learning rate, or batch size.

### Embodiment 155

The apparatus of embodiment 154, wherein the HLA allele type comprises one or more of HLA-A, HLA-B, HLA-C, or a subtype thereof.

### Embodiment 156

The apparatus of embodiment 154, wherein the HLA allele length is from about 8 to about 12 amino acids.

### Embodiment 157

The apparatus of embodiment 155, wherein the HLA allele length is from about 9 to about 11 amino acids.

### Embodiment 158

The polypeptide produced by the apparatus of embodiment 153.

### Embodiment 159

The apparatus of embodiment 153, wherein the polypeptide is a tumor specific antigen.

### Embodiment 160

The apparatus of embodiment 153, wherein the polypeptide comprises an amino acid sequence that specifically binds to an MHC-I protein encoded by a selected MHC allele.

### Embodiment 161

The apparatus of embodiment 153, wherein the positive simulated polypeptide-MHC-I interaction data, the positive real polypeptide-MHC-I interaction data, and the negative real polypeptide-MHC-I interaction data are associated with a selected allele.

### Embodiment 162

The apparatus of embodiment 161, wherein the selected allele is selected from a group consisting of A0201, A0202, A0203, B2703, B2705, and combinations thereof.

### Embodiment 163

The apparatus of embodiment 153, wherein the GAN comprises a Deep Convolutional GAN (DCGAN).

### Embodiment 164

A non-transitory computer readable medium for training a generative adversarial network (GAN), the non-transitory computer readable medium storing processor executable instructions that, when executed by one or more processors, causes the one or more processors to: generate increasingly accurate positive simulated polypeptide-MHC-I interaction data until a GAN discriminator classifies the positive simulated polypeptide-MHC-I interaction data as positive; present the positive simulated polypeptide-MHC-I interaction data, the positive real polypeptide-MHC-I interaction data, and the negative real polypeptide-MHC-I interaction data to a convolutional neural network (CNN), until the CNN classifies polypeptide-MHC-I interaction data as positive or negative; present the positive real polypeptide-MHC-I interaction data and the negative real polypeptide-MHC-I interaction data to the CNN to generate prediction scores; determine, based on the prediction scores, that the GAN is trained; and output the GAN and the CNN.

### Embodiment 165

The non-transitory computer readable medium of embodiment 164, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further cause the one or more processors to: generate, according to a set of GAN parameters, a first simulated dataset comprising simulated positive polypeptide-MHC-I interactions for a WIC allele; combine the first simulated dataset with the positive real polypeptide-MHC-I interactions for the MHC allele, and the negative real polypeptide-MHC-I interactions for the MHC allele to create a GAN training dataset; receive information from a discriminator, wherein the discriminator is configured to determine, according to a decision boundary, whether a positive polypeptide-MHC-I interaction for the WIC allele in the GAN training dataset is positive or negative; adjust, based on accuracy of the information from the discriminator, one or more of the set of GAN parameters or the decision boundary; and repeat a-d until a first stop criterion is satisfied.

### Embodiment 166

The non-transitory computer readable medium of embodiment 165, wherein the processor executable instructions that, when executed by the one or more processors, cause the one or more processors to present the positive simulated polypeptide-MHC-I interaction data, the positive real polypeptide-MHC-I interaction data, and the negative real polypeptide-MHC-I interaction data to a convolutional neural network (CNN), until the CNN classifies polypeptide-MHC-I interaction data as positive or negative further comprise processor executable instructions that, when executed by the one or more processors, cause the one or more processors to: generate, according to the set of GAN parameters, a second simulated dataset comprising simulated positive polypeptide-MHC-I interactions for the MHC allele; combine the second simulated dataset, the positive real polypeptide-MHC-I interaction data, and the negative real polypeptide-MHC-I interaction data for the MHC allele to create a CNN training dataset; present the CNN training dataset to a convolutional neural network (CNN); receive training information from the CNN, wherein the CNN is configured to determine the training information by classifying, according to a set of CNN parameters, a polypeptide-MHC-I interaction for the MHC allele in the CNN training dataset as positive or negative; adjust, based on accuracy of training information, one or more of the set of CNN parameters; and repeat h-j until a second stop criterion is satisfied.

### Embodiment 167

The non-transitory computer readable medium of embodiment 166, wherein the processor executable instructions that, when executed by the one or more processors, cause the one or more processors to present the positive real polypeptide-MHC-I interaction data and the negative real polypeptide-MHC-I interaction data to the CNN to generate prediction scores further comprise processor executable instructions that, when executed by the one or more processors, cause the one or more processors to: present the CNN with the positive real polypeptide-MHC-I interaction data and the negative real polypeptide-MHC-I interaction data, wherein the CNN is further configured to classify, according to the set of CNN parameters, a polypeptide-MHC-I interaction for the MHC allele as positive or negative.

### Embodiment 168

The non-transitory computer readable medium of embodiment 167, wherein the processor executable instructions that, when executed by the one or more processors, cause the one or more processors to determine, based on the prediction scores, that the GAN is trained further comprise processor executable instructions that, when executed by the one or more processors, cause the one or more processors to determine accuracy of the classification of the polypeptide-MHC-I interaction for the MHC allele as positive or negative, and when (if) the accuracy of the classification satisfies a third stop criterion, output the GAN and the CNN.

### Embodiment 169

The non-transitory computer readable medium of embodiment 167, wherein the processor executable instructions that, when executed by the one or more processors, cause the one or more processors to determine, based on the prediction scores, that the GAN is trained further comprise processor executable instructions that, when executed by the one or more processors, cause the one or more processors to determine accuracy of the classification of the polypeptide-MHC-I interaction for the MHC allele as positive or negative, and when (if) the accuracy of the classification does not satisfy a third stop criterion, return to step a.

### Embodiment 170

The non-transitory computer readable medium of embodiment 165, wherein the GAN parameters comprise one or more of allele type, allele length, generating category, model complexity, learning rate, or batch size.

### Embodiment 171

The non-transitory computer readable medium of embodiment 165, wherein the MHC allele is a HLA allele.

### Embodiment 172

The non-transitory computer readable medium of embodiment 171, wherein the HLA allele type comprises one or more of HLA-A, HLA-B, HLA-C, or a subtype thereof.

### Embodiment 173

The non-transitory computer readable medium of embodiment 171, wherein the HLA allele length is from about 8 to about 12 amino acids.

### Embodiment 174

The non-transitory computer readable medium of embodiment 171, wherein the HLA allele length is from about 9 to about 11 amino acids.

### Embodiment 175

The non-transitory computer readable medium of embodiment 164, wherein the processor executable instructions, when executed by the one or more processors, further cause the one or more processors to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesize the polypeptide from the candidate polypeptide-MHC-I interaction that the CNN classifies as a positive polypeptide-MHC-I interaction.

### Embodiment 176

The polypeptide produced by the non-transitory computer readable medium of embodiment 175.

### Embodiment 177

The non-transitory computer readable medium of embodiment 175, wherein the polypeptide is a tumor specific antigen.

### Embodiment 178

The non-transitory computer readable medium of embodiment 175, wherein the polypeptide comprises an amino acid sequence that specifically binds to an MHC-I protein encoded by a selected MHC allele.

### Embodiment 179

The non-transitory computer readable medium of embodiment 164, wherein the positive simulated polypeptide-MHC-I interaction data, the positive real polypeptide-MHC-I interaction data, and the negative real polypeptide-MHC-I interaction data are associated with a selected allele.

### Embodiment 180

The non-transitory computer readable medium of embodiment 179, wherein the selected allele is selected from a group consisting of A0201, A0202, A0203, B2703, B2705, and combinations thereof.

### Embodiment 181

The non-transitory computer readable medium of embodiment 164, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the one or more processors to evaluate a gradient descent expression for the GAN generator.

### Embodiment 182

The non-transitory computer readable medium of embodiment 164, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the one or more processors to: iteratively execute (e.g., optimize) the GAN discriminator in order to increase a likelihood of giving a high probability to positive real polypeptide-MHC-I interaction data and a low probability the positive simulated polypeptide-MHC-I interaction data; and iteratively execute (e.g., optimize) the GAN generator in order to increase a probability of the positive simulated polypeptide-MHC-I interaction data being rated highly.

### Embodiment 183

The non-transitory computer readable medium of embodiment 164, wherein the processor executable instructions that, when executed by the one or more processors, cause the one or more processors to present the positive simulated polypeptide-MHC-I interaction data, the positive real polypeptide-MHC-I interaction data, and the negative real polypeptide-MHC-I interaction data to the convolutional neural network (CNN), until the CNN classifies the polypeptide-MHC-I interaction data as positive or negative real further comprise processor executable instructions that, when executed by the one or more processors, cause the one or more processors to: perform a convolution procedure; perform a Non Linearity (ReLU) procedure; perform a Pooling or Sub Sampling procedure; and perform a Classification (Fully Connected Layer) procedure.

### Embodiment 184

The non-transitory computer readable medium of embodiment 164, wherein the GAN comprises a Deep Convolutional GAN (DCGAN).

### Embodiment 185

The non-transitory computer readable medium of embodiment 165, wherein the first stop criterion comprises an evaluation of a mean squared error (MSE) function.

### Embodiment 186

The non-transitory computer readable medium of embodiment 166, wherein the second stop criterion comprises an evaluation of a mean squared error (MSE) function.

### Embodiment 187

The non-transitory computer readable medium of embodiment 168 or 169, wherein the third stop criterion comprises an evaluation of an area under the curve (AUC) function.

### Embodiment 188

The non-transitory computer readable medium of embodiment 164, wherein the prediction score is a probability of the positive real polypeptide-MHC-I interaction data being classified as positive polypeptide-MHC-I interaction data.

### Embodiment 189

The non-transitory computer readable medium of embodiment 164, wherein the processor executable instructions that, when executed by the one or more processors, cause the one or more processors to determine, based on the prediction scores, that the GAN is trained further comprise processor executable instructions that, when executed by the one or more processors, cause the one or more processors to compare one or more of the prediction scores to a threshold.

### Embodiment 190

A non-transitory computer readable medium for training a generative adversarial network (GAN), the non-transitory computer readable medium storing processor executable instructions that, when executed by one or more processors, causes the one or more processors to: generate increasingly accurate positive simulated polypeptide-MHC-I interaction data until a GAN discriminator classifies the positive simulated polypeptide-MHC-I interaction data as positive; present the positive simulated polypeptide-MHC-I interaction data, the positive real polypeptide-MHC-I interaction data, and the negative real polypeptide-MHC-I interaction data to a convolutional neural network (CNN), until the CNN classifies polypeptide-MHC-I interaction data as positive or negative; present the positive real polypeptide-MHC-I interaction data and the negative real polypeptide-MHC-I interaction data to the CNN to generate prediction scores; determine, based on the prediction scores, that the GAN is not trained; repeat a-c until a determination is made, based on the prediction scores, that the GAN is trained; and output the GAN and the CNN.

### Embodiment 191

The non-transitory computer readable medium of embodiment 190, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the one or more processors to: generate, according to a set of GAN parameters, a first simulated dataset comprising simulated positive polypeptide-MHC-I interactions for an MEW allele; combine the first simulated dataset with the positive real polypeptide-MHC-I interactions for the MHC allele, and the negative real polypeptide-MHC-I interactions for the MHC allele to create a GAN training dataset; receive information from a discriminator, wherein the discriminator is configured to determine, whether a positive polypeptide-MHC-I interaction for the MEW allele in the GAN training dataset is positive or negative; adjust, based on accuracy of the information from the discriminator, one or more of the set of GAN parameters or the decision boundary; and repeat g-j until a first stop criterion is satisfied.

### Embodiment 192

The non-transitory computer readable medium of embodiment 191, wherein the processor executable instructions that, when executed by the one or more processors, cause the apparatus to present the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative further comprise processor executable instructions that, when executed by the one or more processors, cause the one or more processors to: generate, according to the set of GAN parameters, a second simulated dataset comprising simulated positive polypeptide-MHC-I interactions for the MHC allele; combine the second simulated dataset, the positive real polypeptide-MHC-I interaction data, and the negative real polypeptide-MHC-I interaction data for the MEW allele to create a CNN training dataset; present the CNN training dataset to the convolutional neural network (CNN); receive information from the CNN, wherein the CNN is configured to determine the information by classifying, according to a set of CNN parameters, a polypeptide-MHC-I interaction for the MHC allele in the CNN training dataset as positive or negative; adjust, based on accuracy of the information from the CNN, one or more of the set of CNN parameters; and repeat 1-p until a second stop criterion is satisfied.

### Embodiment 193

The non-transitory computer readable medium of embodiment 192, wherein the processor executable instructions that, when executed by the one or more processors, cause the one or more processors to present the positive real polypeptide-MHC-I interaction data and the negative real polypeptide-MHC-I interaction data to the CNN to generate the prediction scores further comprise processor executable instructions that, when executed by the one or more processors, cause the one or more processors to: present the CNN with the positive real polypeptide-MHC-I interaction data and the negative real polypeptide-MHC-I interaction data, wherein the CNN is further configured to classify, according to the set of CNN parameters, a polypeptide-MHC-I interaction for the MHC allele as positive or negative.

### Embodiment 194

The non-transitory computer readable medium of embodiment 193, wherein the processor executable instructions that, when executed by the one or more processors, cause the one or more processors to determine, based on the prediction scores, that the GAN is trained further comprise processor executable instructions that, when executed by the one or more processors, cause the one or more processors to: determine accuracy of the classification by the CNN; determine that the accuracy of the classification satisfies a third stop criterion; and in response to determining that the accuracy of the classification satisfies the third stop criterion, output the GAN and the CNN.

### Embodiment 195

The non-transitory computer readable medium of embodiment 194, wherein the processor executable instructions that, when executed by the one or more processors, cause the one or more processors to determine, based on the prediction scores, that the GAN is trained further comprise processor executable instructions that, when executed by the one or more processors, cause the one or more processors to: determine accuracy of the classification by the CNN; determine that the accuracy of the classification does not satisfy a third stop criterion; and in response to determining that the accuracy of the classification does not satisfy the third stop criterion, returning to step a.

### Embodiment 196

The non-transitory computer readable medium of embodiment 191, wherein the GAN parameters comprise one or more of allele type, allele length, generating category, model complexity, learning rate, or batch size.

### Embodiment 197

The non-transitory computer readable medium of embodiment 191, wherein the MHC allele is an HLA allele.

### Embodiment 198

The non-transitory computer readable medium of embodiment 197, wherein the HLA allele type comprises one or more of HLA-A, HLA-B, HLA-C, or a subtype thereof.

### Embodiment 199

The non-transitory computer readable medium of embodiment 197, wherein the HLA allele length is from about 8 to about 12 amino acids.

### Embodiment 200

The non-transitory computer readable medium of embodiment 197, wherein the HLA allele length is from about 9 to about 11 amino acids.

### Embodiment 201

The non-transitory computer readable medium of embodiment 190, wherein the processor executable instructions, when executed by the one or more processors, further cause the one or more processors to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesize the polypeptide from the candidate polypeptide-MHC-I interaction classified by the CNN as a positive polypeptide-MHC-I interaction.

### Embodiment 202

The polypeptide produced by the non-transitory computer readable medium of embodiment 201.

### Embodiment 203

The non-transitory computer readable medium of embodiment 201, wherein the polypeptide is a tumor specific antigen.

### Embodiment 204

The non-transitory computer readable medium of embodiment 201, wherein the polypeptide comprises an amino acid sequence that specifically binds to an MHC-I protein encoded by a selected MHC allele.

### Embodiment 205

The non-transitory computer readable medium of embodiment 190, wherein the positive simulated polypeptide-MHC-I interaction data, the positive real polypeptide-MHC-I interaction data, and the negative real polypeptide-MHC-I interaction data are associated with a selected allele.

### Embodiment 206

The non-transitory computer readable medium of embodiment 205, wherein the selected allele is selected from a group consisting of A0201, A0202, A0203, B2703, B2705, and combinations thereof.

### Embodiment 207

The non-transitory computer readable medium of embodiment 190, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the one or more processors to evaluate a gradient descent expression for the GAN generator.

### Embodiment 208

The non-transitory computer readable medium of embodiment 190, wherein the processor executable instructions that, when executed by the one or more processors, cause the one or more processors to generate the increasingly accurate positive simulated polypeptide-MHC-I interaction data until the GAN discriminator classifies the positive simulated polypeptide-MHC-I interaction data as positive further comprise processor executable instructions that, when executed by the one or more processors, cause the one or more processors to: iteratively execute (e.g., optimize) the GAN discriminator in order to increase a likelihood of giving a high probability to positive real polypeptide-MHC-I interaction data, a low probability the positive simulated polypeptide-MHC-I interaction data, and a low probability the negative simulated polypeptide-MHC-I interaction data; and iteratively execute (e.g., optimize) the GAN generator in order to increase a probability of the positive simulated polypeptide-MHC-I interaction data being rated highly.

### Embodiment 209

The non-transitory computer readable medium of embodiment 190, wherein the processor executable instructions that, when executed by the one or more processors, cause the one or more processors to present the positive simulated polypeptide-MHC-I interaction data, the positive real polypeptide-MHC-I interaction data, and the negative real polypeptide-MHC-I interaction data to the convolutional neural network (CNN), until the CNN classifies polypeptide-MHC-I interaction data as positive or negative further comprise processor executable instructions that, when executed by the one or more processors, cause the one or more processors to: perform a convolution procedure; perform a Non Linearity (ReLU) procedure; perform a Pooling or Sub Sampling procedure; and perform a Classification (Fully Connected Layer) procedure.

### Embodiment 210

The non-transitory computer readable medium of embodiment 190, wherein the GAN comprises a Deep Convolutional GAN (DCGAN).

### Embodiment 211

The non-transitory computer readable medium of embodiment 191, wherein the first stop criterion comprises an evaluation of a mean squared error (MSE) function.

### Embodiment 212

The non-transitory computer readable medium of embodiment 190, wherein the second stop criterion comprises an evaluation of a mean squared error (MSE) function.

### Embodiment 213

The non-transitory computer readable medium of embodiment 194 or 195, wherein the third stop criterion comprises an evaluation of an area under the curve (AUC) function.

### Embodiment 214

The non-transitory computer readable medium of embodiment 190, wherein the prediction score is a probability of the positive real polypeptide-MHC-I interaction data being classified as positive polypeptide-MHC-I interaction data.

### Embodiment 215

The non-transitory computer readable medium of embodiment 190, wherein the processor executable instructions that, when executed by the one or more processors, cause the one or more processors to determine, based on the prediction scores, that the GAN is trained further comprise processor executable instructions that, when executed by the one or more processors, cause the one or more processors to compare one or more of the prediction scores to a threshold.

### Embodiment 216

A non-transitory computer readable medium for training a generative adversarial network (GAN), the non-transitory computer readable medium storing processor executable instructions that, when executed by one or more processors, causes the one or more processors to: generate, according to a set of GAN parameters, a first simulated dataset comprising simulated positive polypeptide-MHC-I interactions for an MEW allele; combine the first simulated dataset with the positive real polypeptide-MHC-I interactions for the MHC allele, and the negative real polypeptide-MHC-I interactions for the MHC allele to create a GAN training dataset; receive information from a discriminator, wherein the discriminator is configured to determine, according to a decision boundary, whether a positive polypeptide-MHC-I interaction for the MEW allele in the GAN training dataset is positive or negative; adjust, based on accuracy of the information from the discriminator, one or more of the set of GAN parameters or the decision boundary; repeat a-d until a first stop criterion is satisfied; generate, by the GAN generator according to the set of GAN parameters, a second simulated dataset comprising simulated positive polypeptide-MHC-I interactions for the MHC allele; combine the second simulated dataset, the positive real polypeptide-MHC-I interaction data, and the negative real polypeptide-MHC-I interaction data for the MEW allele to create a CNN training dataset; present the CNN training dataset to a convolutional neural network (CNN); receive training information from the CNN, wherein the CNN is configured to determine the training information by classifying, according to a set of CNN parameters, a polypeptide-MHC-I interaction for the MHC allele in the CNN training dataset as positive or negative; adjust, based on accuracy of the training information, one or more of the set of CNN parameters; repeat h-j until a second stop criterion is satisfied; present the CNN with the positive real polypeptide-MHC-I interaction data and the negative real polypeptide-MHC-I interaction data; receive training information from the CNN, wherein the CNN is configured to determine the training information by classifying, according to the set of CNN parameters, a polypeptide-MHC-I interaction for the MHC allele as positive or negative; and determine accuracy of the training information, wherein when (if) the accuracy of the training information satisfies a third stop criterion, outputting the GAN and the CNN, wherein when (if) the accuracy of the training information does not satisfy the third stop criterion, returning to step a.

### Embodiment 217

The non-transitory computer readable medium of embodiment 216, wherein the GAN parameters comprise one or more of allele type, allele length, generating category, model complexity, learning rate, or batch size.

### Embodiment 218

The non-transitory computer readable medium of embodiment 216, wherein the MHC allele is an HLA allele.

### Embodiment 219

The non-transitory computer readable medium of embodiment 218, wherein the HLA allele type comprises one or more of HLA-A, HLA-B, HLA-C, or a subtype thereof.

### Embodiment 220

The non-transitory computer readable medium of embodiment 218, wherein the HLA allele length is from about 8 to about 12 amino acids.

### Embodiment 221

The non-transitory computer readable medium of embodiment 218, wherein the HLA allele length is from about 9 to about 11 amino acids.

### Embodiment 222

The non-transitory computer readable medium of embodiment 216, wherein the processor executable instructions, when executed by the one or more processors, further cause the one or more processors to: present a dataset to the CNN, wherein the dataset comprises a plurality of candidate polypeptide-MHC-I interactions, wherein the CNN is further configured to classify each of the plurality of candidate polypeptide-MHC-I interactions as a positive or a negative polypeptide-MHC-I interaction; and synthesizing the polypeptide from the candidate polypeptide-MHC-I interaction classified by the CNN as a positive polypeptide-MHC-I interaction.

### Embodiment 223

The polypeptide produced by the non-transitory computer readable medium of embodiment 222.

### Embodiment 224

The non-transitory computer readable medium of embodiment 222, wherein the polypeptide is a tumor specific antigen.

### Embodiment 225

The non-transitory computer readable medium of embodiment 222, wherein the polypeptide comprises an amino acid sequence that specifically binds to an MHC-I protein encoded by a selected MHC allele.

### Embodiment 226

The non-transitory computer readable medium of embodiment 216, wherein the positive simulated polypeptide-MHC-I interaction data, the positive real polypeptide-MHC-I interaction data, and the negative real polypeptide-MHC-I interaction data are associated with a selected allele.

### Embodiment 227

The non-transitory computer readable medium of embodiment 226, wherein the selected allele is selected from a group consisting of A0201, A0202, A0203, B2703, B2705, and combinations thereof.

### Embodiment 228

The non-transitory computer readable medium of embodiment 216, wherein the processor executable instructions that, when executed by the one or more processors, cause the one or more processors to repeat a-d until the first stop criterion is satisfied further comprise processor executable instructions that, when executed by the one or more processors, cause the one or more processors to evaluate a gradient descent expression for the GAN generator.

### Embodiment 229

The non-transitory computer readable medium of embodiment 216, wherein the processor executable instructions that, when executed by the one or more processors, cause the one or more processors to repeat a-d until the first stop criterion is satisfied further comprise processor executable instructions that, when executed by the one or more processors, cause the one or more processors to: iteratively execute (e.g., optimize) the GAN discriminator in order to increase a likelihood of giving a high probability to positive real polypeptide-MHC-I interaction data, a low probability the positive simulated polypeptide-MHC-I interaction data, and a low probability the negative simulated polypeptide-MHC-I interaction data; and iteratively execute (e.g., optimize) the GAN generator in order to increase a probability of the positive simulated polypeptide-MHC-I interaction data being rated highly.

### Embodiment 230

The non-transitory computer readable medium of embodiment 216, wherein the processor executable instructions that, when executed by the one or more processors, cause the one or more processors to present the CNN training dataset to the CNN further comprise processor executable instructions that, when executed by the one or more processors, cause the one or more processors to: perform a convolution procedure; perform a Non Linearity (ReLU) procedure; perform a Pooling or Sub Sampling procedure; and perform a Classification (Fully Connected Layer) procedure.

### Embodiment 231

The non-transitory computer readable medium of embodiment 216, wherein the GAN comprises a Deep Convolutional GAN (DCGAN).

### Embodiment 232

The non-transitory computer readable medium of embodiment 216, wherein the first stop criterion comprises an evaluation of a mean squared error (MSE) function.

### Embodiment 233

The non-transitory computer readable medium of embodiment 216, wherein the second stop criterion comprises an evaluation of a mean squared error (MSE) function.

### Embodiment 234

The non-transitory computer readable medium of embodiment 216, wherein the third stop criterion comprises an evaluation of an area under the curve (AUC) function.

### Embodiment 235

A non-transitory computer readable medium for training a generative adversarial network (GAN), the non-transitory computer readable medium storing processor executable instructions that, when executed by one or more processors, causes the one or more processors to: train a convolutional neural network (CNN) by the same means as the apparatus of embodiment 83; present a dataset to the CNN, wherein the dataset comprises a plurality of candidate polypeptide-MHC-I interactions, wherein the CNN is configured to classify each of the plurality of candidate polypeptide-MHC-I interactions as a positive or a negative polypeptide-MHC-I interaction; and synthesize a polypeptide associated with a candidate polypeptide-MHC-I interaction classified by the CNN as a positive polypeptide-MHC-I interaction.

### Embodiment 236

The non-transitory computer readable medium of embodiment 235, wherein the CNN is trained based on one or more GAN parameters comprising one or more of allele type, allele length, generating category, model complexity, learning rate, or batch size.

### Embodiment 237

The non-transitory computer readable medium of embodiment 236, wherein the HLA allele type comprises one or more of HLA-A, HLA-B, HLA-C, or a subtype thereof.

### Embodiment 238

The non-transitory computer readable medium of embodiment 236, wherein the HLA allele length is from about 8 to about 12 amino acids.

### Embodiment 239

The non-transitory computer readable medium of embodiment 236, wherein the HLA allele length is from about 9 to about 11 amino acids.

### Embodiment 240

The polypeptide produced by the non-transitory computer readable medium of embodiment 235.

### Embodiment 241

The non-transitory computer readable medium of embodiment 235, wherein the polypeptide is a tumor specific antigen.

### Embodiment 242

The non-transitory computer readable medium of embodiment 235, wherein the polypeptide comprises an amino acid sequence that specifically binds to an MHC-I protein encoded by a selected human leukocyte antigen (HLA) allele.

### Embodiment 243

The non-transitory computer readable medium of embodiment 235, wherein the positive simulated polypeptide-MHC-I interaction data, the positive real polypeptide-MHC-I interaction data, and the negative real polypeptide-MHC-I interaction data are associated with a selected allele.

### Embodiment 244

The non-transitory computer readable medium of embodiment 243, wherein the selected allele is selected from a group consisting of A0201, A0202, A0203, B2703, B2705, and combinations thereof.

### Embodiment 245

The non-transitory computer readable medium of embodiment 235, wherein the GAN comprises a Deep Convolutional GAN (DCGAN).

We claim:

1. A method for training a generative adversarial network (GAN), comprising:
a. generating, by a GAN generator, increasingly accurate positive simulated data until a GAN discriminator classifies the positive simulated data as positive;
b. presenting the positive simulated data, positive real data, and negative real data to a convolutional neural network (CNN), until the CNN classifies each type of data as positive or negative;
c. presenting the positive real data and the negative real data to the CNN to generate prediction scores; and
d. determining, based on the prediction scores, whether the GAN is trained or not trained, and when the GAN is not trained, repeating steps a-c until a determination is made, based on the prediction scores, that the GAN is trained.

2. The method of claim 1, wherein the positive simulated data, the positive real data, and the negative real data comprise biological data.

3. The method of claim 1, wherein the positive simulated data comprises positive simulated polypeptide-major histocompatibility complex class I (MHC-I) interaction data, the positive real data comprises positive real polypeptide-MHC-I interaction data, and the negative real data comprises negative real polypeptide-MHC-I interaction data.

4. The method of claim 3, wherein generating the increasingly accurate positive simulated polypeptide-MHC-I interaction data until the GAN discriminator classifies the positive simulated polypeptide-MHC-I interaction data as real comprises:
e. generating, by the GAN generator according to a set of GAN parameters, a first simulated dataset comprising simulated positive polypeptide-MHC-I interactions for a MHC allele;
f. combining the first simulated dataset with the positive real polypeptide-MHC-I interactions for the MHC allele, and the negative real polypeptide-MHC-I interactions for the MHC allele to create a GAN training dataset;
g. determining, by a discriminator according to a decision boundary, whether a respective polypeptide-MHC-I interaction for the MHC allele in the GAN training dataset is simulated positive, real positive, or real negative;
h. adjusting, based on accuracy of the determination by the discriminator, one or more of the set of GAN parameters or the decision boundary; and
i. repeating steps e-h until a first stop criterion is satisfied.

5. The method of claim 4, wherein presenting the positive simulated polypeptide-MHC-I interaction data, the positive real polypeptide-MHC-I interaction data, and the negative real polypeptide-MHC-I interaction data to the convolutional neural network (CNN), until the CNN classifies respective polypeptide-MHC-I interaction data as positive or negative comprises:
j. generating, by the GAN generator according to the set of GAN parameters, a second simulated dataset comprising simulated positive polypeptide-MHC-I interactions for the MHC allele;
k. combining the second simulated dataset, the positive real polypeptide-MHC-I interactions for the MHC allele, and the negative real polypeptide-MHC-I interactions for the MHC allele to create a CNN training dataset;
l. presenting the CNN training dataset to the convolutional neural network (CNN);
m. classifying, by the CNN according to a set of CNN parameters, a respective polypeptide-MHC-I interaction for the MHC allele in the CNN training dataset as positive or negative;
n. adjusting, based on accuracy of the classification by the CNN, one or more of the set of CNN parameters; and
o. repeating steps 1-n until a second stop criterion is satisfied.

6. The method of claim 5, wherein presenting the positive real polypeptide-MHC-I interaction data and the negative real polypeptide-MHC-I interaction data to the CNN to generate prediction scores comprises:
classifying, by the CNN according to the set of CNN parameters, a respective polypeptide-MHC-I interaction for the MHC allele as positive or negative.

7. The method of claim 6, wherein determining, based on the prediction scores, whether the GAN is trained comprises determining accuracy of the classification by the CNN, wherein when the accuracy of the classification satisfies a third stop criterion, outputting the GAN and the CNN.

8. The method of claim 6, wherein determining, based on the prediction scores, whether the GAN is trained comprises determining accuracy of the classification by the CNN, wherein when the accuracy of the classification does not satisfy a third stop criterion, returning to step a.

9. The method of claim 4, wherein the GAN parameters comprise one or more of allele type, allele length, generating category, model complexity, learning rate, or batch size.

10. The method of claim 9, wherein the allele type comprises one or more of HLA-A, HLA-B, HLA-C, or a subtype thereof.

11. The method of claim 9, wherein the allele length is from about 8 to about 12 amino acids.

12. The method of claim 11, wherein the allele length is from about 9 to about 11 amino acids.

13. The method of claim 3, further comprising:
presenting a dataset to the CNN, wherein the dataset comprises a plurality of candidate polypeptide-MHC-I interactions;
classifying, by the CNN, each of the plurality of candidate polypeptide-MHC-I interactions as a positive or a negative polypeptide-MHC-I interaction; and
synthesizing the polypeptide from the candidate polypeptide-MHC-I interaction classified as a positive polypeptide-MHC-I interaction.

14. The method of claim 13, wherein the polypeptide comprises an amino acid sequence that specifically binds to an MHC-I protein encoded by a selected MHC allele.

15. The method of claim 3, wherein the positive simulated polypeptide-MHC-I interaction data, the positive real polypeptide-MHC-I interaction data, and the negative real polypeptide-MHC-I interaction data are associated with a selected allele.

16. The method of claim 17, wherein the selected allele is selected from a group consisting of A0201, A0202, A0203, B2703, B2705, and combinations thereof.

17. The method of claim 3, wherein generating the increasingly accurate positive simulated polypeptide-MHC-I interaction data until the GAN discriminator classifies the positive simulated polypeptide-MHC-I interaction data as positive comprises evaluating a gradient descent expression for the GAN generator.

18. The method of claim 3, wherein generating the increasingly accurate positive simulated polypeptide-MHC-I interaction data until the GAN discriminator classifies the positive simulated polypeptide-MHC-I interaction data as positive comprises:
iteratively executing the GAN discriminator in order to increase a likelihood of giving a high probability to positive real polypeptide-MHC-I interaction data, a low probability to the positive simulated polypeptide-MHC-I interaction data, and a low probability to the negative real polypeptide-MHC-I interaction data; and
iteratively executing the GAN generator in order to increase a probability of the positive simulated polypeptide-MHC-I interaction data being rated highly.

19. The method of claim 8, wherein the first stop criterion comprises evaluating a mean squared error (MSE) function, the second stop criterion comprises evaluating a mean squared error (MSE) function, and the third stop criterion comprises evaluating an area under the curve (AUC) function.

20. The method of claim 1, further comprising outputting the GAN and the CNN.
